(19) United States
(12) Patent Application Publication
(10) Pub. No.: US 2009/0220607 A1
(43) Pub. Date: Sep. 3, 2009

(54) POLYMERIC COMPOSITIONS AND METHODS OF MAKING AND USING THEREOF

(76) Inventors: Patrick F. Kiser, Salt Lake City, UT (US); Meredith C. Roberts, Salt Lake City, UT (US)

Correspondence Address: Ballard Spahr Andrews & Ingersoll, LLP, SUITE 1000, 999 PEACHTREE STREET, ATLANTA, GA, 30309-3915, US

(21) Appl. No.: 12/066,941

(22) PCT Filed: Sep. 11, 2006
(86) PCT No.: PCT/US2006/035235

§ 371 Date: Oct. 15, 2008

Related U.S. Application Data
(60) Provisional application No. 60/717,528, filed on Sep. 15, 2005.

(51) Int. Cl. A61L 27/54; A61L 27/52; A61P 17/02; A61K 39/395; A61K 38/02; A61L 29/16; A61L 31/16; C08G 63/91

(52) U.S. Cl. 424/487; 525/437; 424/486; 424/130.1; 514/2

(57) ABSTRACT

Described herein are polymeric compositions having a polymer residue and a crosslinker residue, wherein the polymer residue is bonded to the crosslinker residue with a moiety formed from a cycloaddition reaction. Also, described are methods of making and using such polymeric compositions.

This application claims priority to U.S. Provisional Application Ser. No. 60/717,528, filed Sep. 15, 2005, which is herein incorporated by this reference in its entirety.

### ACKNOWLEDGEMENTS

The research leading to this invention was funded in part by the National Institutes of Health, Grant No. NIH-NIAID R21 AI6262445-01. The U.S. Government may have certain rights in this invention.

### BACKGROUND

Polymeric compositions are widely used in medical applications. For example, various polymers have been used as suture materials and for fracture fixation (see e.g., U.S. Pat. Nos. 5,902,599 and 5,837,752). Polymers have also been used in polymer-based drug delivery systems. For drug delivery, polymers are typically used as a matrix for the controlled or sustained release of biologically active agents. Examples of such polymer-based drug delivery systems are described in, for example, U.S. Pat. Nos. 6,183,781, 6,110,503, 5,989,463, 5,916,598, 5,817,343, and 5,650,173. Polymers have also been used as scaffolds for tissue engineering (see e.g., U.S. Pat. No. 6,103,255). Additionally, polymers have been used in dental applications as adhesives and fillers (see e.g., U.S. Pat. No. 5,902,599).

One type of polymeric composition that has received considerable attention for medical applications is the hydrogel. Hydrogels are three-dimensional polymer networks composed of homopolymers or copolymers that are capable of absorbing large amounts of water. Thus, a characteristic of hydrogels is that they swell in water or aqueous fluids without dissolving. Their high water content and soft consistency make hydrogels similar to natural living tissue more than any other class of synthetic biomaterials. Accordingly, many hydrogels are compatible with living systems and hydrogels have found numerous applications in medical and pharmaceutical industries. For example, hydrogels have been investigated widely as drug carriers due to their adjustable swelling capacities, which permit flexible control of drug release rates.

Under certain situations, it may be desirable to prepare a polymeric composition at the site of its intended use. However, a disadvantage of some polymeric compositions is that the polymers must be formed before they can be used. This is because the preparation of many types of polymers typically requires extreme conditions that are not compatible with the environment that the polymeric composition is intended to be used in (e.g., uses in biological systems). For example, the preparation of some polymers can require high temperature, exotic reagents, initiators, and/or solvents, and expensive and/or toxic catalysts. Another reason for preparing a polymeric composition before it can be used is that polymers are typically prepared from reactive monomers or oligomers, which, instead of forming the desired polymer network, can react with cells, tissues, biomolecules, and other species present in a given application.

Similar problems also exist when using polymeric compositions that require crosslinking, which is the formation of a linkage (e.g., covalent, non-covalent, or combinations thereof) between polymer chains or between portions of the same polymer chain. Crosslinking is frequently accomplished through the introduction of a crosslinker that has functionality capable of reacting chemically with functionality on one or more polymer chains. Crosslinking is often done to provide rigidity to the polymer system. For hydrogels, the polymer network is created by forming crosslinks between polymeric chains. For many polymeric compositions, extreme conditions and reactive crosslinkers are required for crosslinking. And as discussed above, such conditions are not generally compatible with certain environments (e.g., biological systems). Thus, crosslinking is often performed prior to using a polymer composition in a given application.

The wide variety of medical applications for polymeric compositions demonstrates the need for the development of different types of compositions with varying physical properties for use in various applications (e.g., medical applications). Further it would desirable to have polymeric compositions that could be prepared or crosslinked in situ in a biological environment under mild conditions. The subject matter disclosed herein meets these and other needs.

### SUMMARY

In accordance with the purposes of the disclosed materials, compounds, compositions, articles, devices, and methods, as embodied and broadly described herein, the disclosed subject matter, in one aspect, relates to compounds and compositions and methods for preparing and using such compounds and compositions. In a further aspect, disclosed herein are polymeric compositions comprising a polymer residue and a crosslinker residue, wherein the polymer residue is bonded to the crosslinker residue with a moiety formed from a cycloaddition reaction. In still a further aspect, disclosed herein are methods of making and using such polymeric compositions.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### DESCRIPTION OF THE FIGURES

The accompanying figures, which are incorporated in and constitute a part of this specification, illustrate several aspects described below.

FIG. 1 is a schematic of in situ gelation using click chemistry. A crosslinked hydrogel can be formed in water using an azide-functionalized, multi-branched and hydrophilic polymer, such as 4-arm PEG, and a hydrophilic dialkyne crosslinker. This triazole-forming cycloaddition reaction can use copper(I) catalyst or be catalyst-free (e.g., by using electron-deficient alkynes).

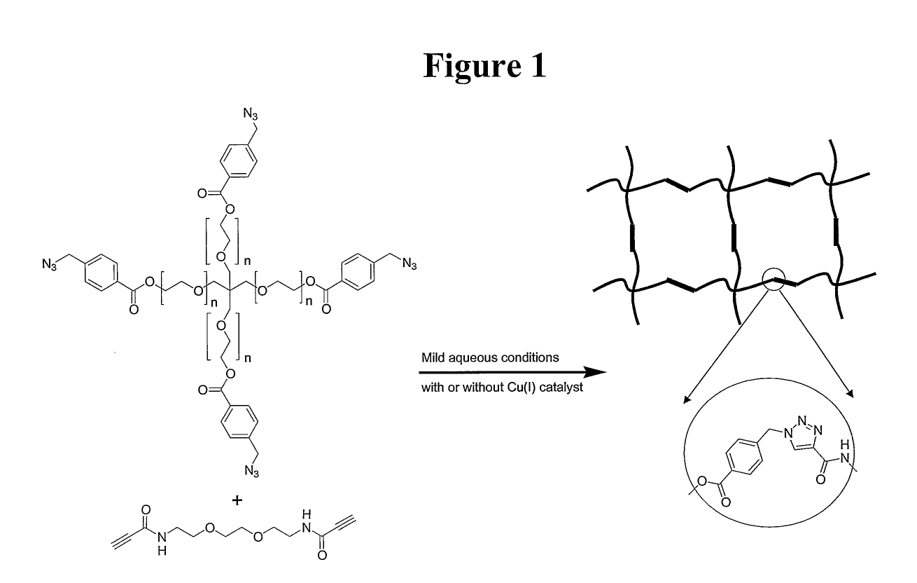

FIG. 2 is a group of schemes for polymer and crosslinker syntheses. Scheme 2A shows the synthesis of azidotoluic acid, which was synthesized prior to functionalizing 4-arm PEG (Scheme 2B). Syntheses of dialkyne and dialkene crosslinkers are shown for dipentynoic ester PEG (Scheme C), dipropiolic amide PEG (Scheme D), and dinorbornene ester PEG (Scheme E).

FIG. 3 is a pair of photographs showing hydrogel formation by catalyzed click chemistry and catalyst-free click chemistry. The left photograph is a representative image of a traditional click chemistry-based hydrogel formed using 0.0169 M azide-functionalized 4-arm PEG, 0.0338 M di(pentynoic ester) PEG crosslinker, 0.00169 M copper (II) sulfate, and 0.0169 M sodium ascorbate in water. The gelation formed within 15 minutes incubation at 37° C. The right photograph is a representative image of a catalyst-free click hydrogel formed using 0.169 M azide-functionalized 4-arm PEG and 0.338 M di(propiolic amide) ethylene glycol (chemical structures shown in FIG. 2) following 48 hours incubation at 37° C. in water.

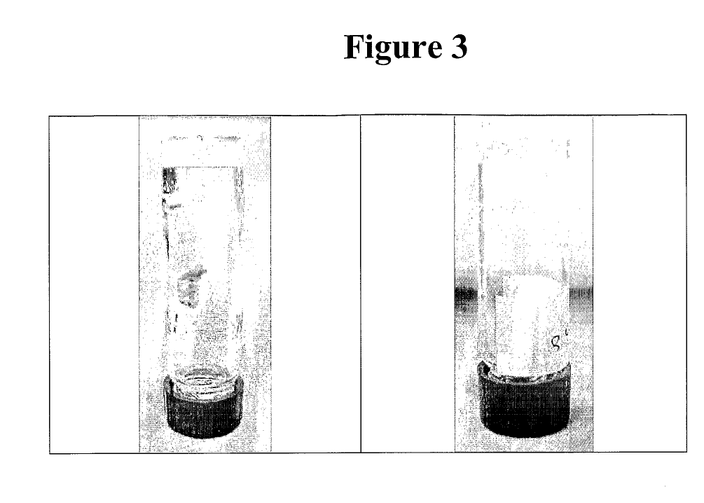

FIG. 4 is a scheme showing the synthesis of a cyclooctyne-functionalized crosslinker. This cyclic-strained alkyne crosslinker can promote the formation of catalyst-free click hydrogels with multivalent azide-functionalized polymers, similar to that seen with the cyclic-strained norbornene crosslinker (e.g., Scheme E of FIG. 2).

### DETAILED DESCRIPTION

The materials, compounds, compositions, articles, devices, and methods described herein may be understood more readily by reference to the following detailed description of specific aspects of the disclosed subject matter and the Examples included therein and to the Figures.

Before the present materials, compounds, compositions, articles, devices, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### DEFINITIONS

In this specification and in the claims that follow, reference will be made to a number of terms that shall be defined to have the following meanings:

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the polymer” includes mixtures of two or more such polymers, and the like.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value,” and possible ranges between values are also disclosed, as appropriately understood by the skilled artisan. For example, if the value “10” is disclosed, then “less than or equal to 10” as well as “greater than or equal to 10” is also disclosed. It is also understood that throughout the application data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

References in the specification and concluding claims to parts by weight of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

A “residue” of a chemical species, as used in the specification and concluding claims, refers to the moiety that is the resulting product of the chemical species in a particular reaction scheme or subsequent formulation or chemical product, regardless of whether the moiety is actually obtained from the chemical species.

“A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “alkyl” as used herein is a branched or unbranched saturated hydrocarbon group of 1 to 24 carbon atoms, such as methyl, ethyl, n-propyl; isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, substituted or unsubstituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein. A “lower alkyl” group is an alkyl group containing from one to six carbon atoms.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, substituted or unsubstituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “polyalkylene group” as used herein is a group having two or more CH₂ groups linked to one another. The polyalkylene group can be represented by the formula —(CH₂)ₐ—, where “a” is an integer of from 2 to 500.

The term “alkoxy” as used herein is an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This may be presumed in structural formulae herein wherein an asymmetric alkene is present, or it may be explicitly indicated by the bond symbol C═C. The alkenyl group can be substituted with one or more groups including, but not limited to, substituted or unsubstituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, substituted or unsubstituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, substituted or unsubstituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, substituted or unsubstituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aryl” as used herein is a group that contains any carbon-based aromatic group including, but not limited to, benzene, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, substituted or unsubstituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein. The term “biaryl” is a specific type of aryl group and is included in the definition of “aryl.” Biaryl refers to two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula NA¹A²A³, where A¹, A², and A³ can be, independently, hydrogen or substituted or unsubstituted alkyl, cycloalkyl, alkenyl, cycloalkenyl, alkynyl, cycloalkynyl, aryl, or heteroaryl group described above.

The term “carboxylic acid” as used herein is represented by the formula —C(O)OH.

The term “ester” as used herein is represented by the formula —OC(O)A¹ or —C(O)OA¹, where A¹ can be a substituted or unsubstituted alkyl, cycloalkyl, alkenyl, cycloalkenyl, alkynyl, cycloalkynyl, aryl, or heteroaryl group described above. The term “polyester” as used herein is represented by the formula -(A¹O(O)C-A²-C(O)O)ₐ— or -(A¹O(O)C-A²-OC(O))ₐ—, where A¹ and A² can be, independently, a substituted or unsubstituted alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, a substituted or unsubstituted alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, a substituted or unsubstituted alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The term “halide” as used herein refers to the halogens fluorine, chlorine, bromine, and iodine.

The term “hydroxyl” as used herein is represented by the formula —OH.

The term “ketone” as used herein is represented by the formula A¹C(O)A², where A¹ and A² can be, independently, a substituted or unsubstituted alkyl, cycloalkyl, alkenyl, cycloalkenyl, alkynyl, cycloalkynyl, aryl, or heteroaryl group described above.

The term “azide” as used herein is represented by the formula —N₃.

The term “nitro” as used herein is represented by the formula —NO₂.

The term “nitrile” as used herein is represented by the formula —CN.

The term “silyl” as used herein is represented by the formula —SiA¹A²A³, where A¹, A², and A³ can be, independently, hydrogen or a substituted or unsubstituted alkyl, cycloalkyl, alkoxy, alkenyl, cycloalkenyl, alkynyl, cycloalkynyl, aryl, or heteroaryl group described above.

The term “sulfo-oxo” as used herein is represented by the formulas —S(O)A¹, S(O)₂A¹, —OS(O)₂A¹, or —OS(O)₂OA¹, where A¹ can be hydrogen or a substituted or unsubstituted alkyl, cycloalkyl, alkenyl, cycloalkenyl, alkynyl, cycloalkynyl, aryl, or heteroaryl group described above. Throughout this specification “S(O)” is a short hand notation for S═O. The term “sulfonyl” is used herein to refer to the sulfo-oxo group represented by the formula —S(O)₂A¹, where A¹ can be hydrogen or a substituted or unsubstituted alkyl, cycloalkyl, alkenyl, cycloalkenyl, alkynyl, cycloalkynyl, aryl, or heteroaryl group described above. The term “sulfone” as used herein is represented by the formula A¹S(O)₂A², where A¹ and A² can be, independently, a substituted or unsubstituted alkyl, cycloalkyl, alkenyl, cycloalkenyl, alkynyl, cycloalkynyl, aryl, or heteroaryl group described above. The term “sulfoxide” as used herein is represented by the formula A¹S(O)A², where A¹ and A² can be, independently, a substituted or unsubstituted alkyl, cycloalkyl, alkenyl, cycloalkenyl, alkynyl, cycloalkynyl, aryl, or heteroaryl group described above.

The term “thiol” as used herein is represented by the formula —SH.

“R,” “R′,” “L,” “L′,” “X,” “Y,” and “Z” as used herein can, independently, possess one or more of the groups listed above. For example, if R′ is a polyether group, one of the hydrogen atoms of the polyether group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “a polyether group comprising an alkene group,” the alkene group can be incorporated within the backbone of the polyether group. Alternatively, the alkene group can be attached to the backbone of the polyether group. The nature of the group(s) that is (are) selected will determine if the first group is embedded or attached to the second group.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture.

Reference will now be made in detail to specific aspects of the disclosed materials, compounds, compositions, articles, and methods, examples of which are illustrated in the accompanying Examples and Figures.

### Compositions

Disclosed herein are materials, compounds, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a composition is disclosed and a number of modifications that can be made to a number of components of the composition are discussed, each and every combination and permutation that are possible are specifically contemplated unless specifically indicated to the contrary. Thus, if a class of components or moieties A, B, and C are disclosed as well as a class of components or moieties D, E, and F and an example of a composition A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific aspect or combination of aspects of the disclosed methods, and that each such combination is specifically contemplated and should be considered disclosed.

In one aspect, disclosed herein are polymeric compositions comprising a hydrophilic polymer residue and a crosslinker residue, wherein the hydrophilic polymer residue is bonded to the crosslinker residue with a moiety formed from a cycloaddition reaction. In many examples disclosed herein, the hydrophilic polymer residue can be bonded to the crosslinker residue with a moiety formed from a 3+2 or 2+2 cycloaddition reaction. The disclosed polymeric compositions can also be prepared in situ under mild aqueous conditions, as is described herein.

In some examples, the disclosed polymeric composition can comprises one or more moieties having Formula I:

L-(Z-R)ₙ (I)

where L is a residue of a crosslinker, R is a residue of a hydrophilic polymer, Z is a moiety formed from a cycloaddition reaction, and n is at least 2. In other examples, n is 3, 4, 5, 6, 7, 8, 9, 10, or greater than 10, where any of the stated values can form an upper and/or lower endpoint when appropriate.

Formula I represents a crosslinking structure present in the disclosed polymeric compositions. In this crosslinking structure, Z is a link between a crosslinker residue, L, and a hydrophilic polymer residue, R. The crosslinking structure illustrated by Formula I can be formed by the methods disclosed herein.

Generally, the hydrophilic polymer residue, R, of the disclosed polymeric compositions is derived from a hydrophilic polymer, denoted R′. As disclosed herein, the hydrophilic polymer R′ comprises one or more cycloaddition reactive moieties, denoted X. Similarly, the crosslinker residue, L, is derived from a crosslinker, denoted L′, which, as is disclosed herein, comprises two or more cycloaddition reaction moieties, denoted Y. When the hydrophilic polymer with its one or more cycloaddition reactive moieties (denoted as R′-X) and the crosslinker with its two or more cycloaddition reactive moieties (denoted as L′-Yₙ) are reacted together, the cycloaddition reactive moieties, X and Y, undergo a cycloaddition reaction to produce the moiety Z in Formula I above. Thus, Z links the remaining residue of the hydrophilic polymer, i.e., R, to the remaining residue of the crosslinker, i.e., L. This general reaction scheme (Scheme 1) can be illustrated as follows:

Scheme 1

R′-X+L′-(Y)ₙ→L-(Z-R)ₙ

While the hydrophilic polymer R′ is shown with one X substituent in Scheme 1, it is understood that more than on X can, and often will, be present on R′. Further Scheme 1 is empirical only and is not meant to imply a 1 to 1 stoichiometric relationship between the crosslinker and hydrophilic polymer. More than one hydrophilic polymer can react with more than one crosslinker. Also, more than one crosslinker can react with the same hydrophilic polymer molecule. Alternatively, more than one hydrophilic polymer molecule can react with the same crosslinker molecule.

In the disclosed polymeric compositions, if L is a residue of divalent crosslinker (i.e., the crosslinker L′ contained two cycloaddition reactive moieties, Y, that formed bonds with a cycloaddition reactive moiety, X, on a hydrophilic polymer, R′), then n will be 2. Similarly, if L is a residue of trivalent crosslinker, then n will be 3, and so forth. In certain examples, disclosed herein are polymeric compositions where crosslinker residue, L, is a residue of a di-, tri-, tetra-, penta-, hexa-, hepta-, octa-, nona-, or deca-valent crosslinker. In reference to Formula I, disclosed herein are polymeric compositions where n is 2, 3, 4, 5, 6, 7, 8, 9, or 10.

In some examples of the disclosed polymeric compositions, there can be one moiety having Formula I. In this situation, the polymeric composition can be said to have one crosslinking structure whereby a crosslinker residue, L, is linked to a hydrophilic polymer residue, R, with a moiety, Z, formed by a cycloaddition reaction. However, there are typically multiple crosslinking structures represented by Formula I in the disclosed polymeric compositions. Such compositions can be a network of multiple hydrophilic polymer residues, R, linked to multiple crosslinker residues, L, with a cycloaddition reaction. Such polymeric compositions can comprise a hydrogel. It is also contemplated that other types of crosslinking structures can be present in the disclosed polymeric compositions.

The polymeric compositions described herein can assume numerous shapes and forms depending upon the intended end-use. In one example, the composition is a laminate, a gel, a bead, a sponge, a film, a mesh, or a matrix. The procedures disclosed in U.S. Pat. Nos. 6,534,591 and 6,548,081, which are incorporated by reference in their entireties, can be used for preparing polymeric compositions having different forms.

The polymeric compositions disclosed herein can also be biodegradable. For example, the disclosed polymeric compositions can be biodegradable by peptides such as naturally occurring enzymes that can degrade the polymeric compositions over time.

In other examples, the polymeric compositions disclosed herein are not products of a cycloaddition based conjugation. Conjugation occurs when one component is bonded to another, without crosslinking of multiple components. Such conjugation can be illustrated by the following structure: A¹-Z-A², where A¹ and A² are different and Z is, for example, a moiety formed from a cycloaddition reaction. Also, in some example, the polymeric composition is not a polyacrylamide or polyacrylamide hydrogel crosslinked with a photoactivated 2+2 cycloaddition.

Hydrophilic Polymer and Residue Thereof

The hydrophilic polymer, R′, and likewise the residue derived therefrom, R, can be any polymeric compound where all or a portion of the compound is hydrophilic. By “hydrophilic” is meant that the polymer or residue thereof is soluble at greater than about 1 mg/L of water. For example, a hydrophilic polymer or residue thereof can be soluble at about 5 mg/L, 10 mg/L, 50 mg/L, 100 mg/L, 500 mg/L, or greater than 1 g/L. For example, a hydrophilic polymer or residue thereof can comprise a homopolymer or a copolymer (e.g., a block, graft, or graft comb copolymer) where one or more of the polymer blocks comprise a hydrophilic segment. Suitable hydrophilic polymers and residues thereof can be obtained from commercial sources or can be prepared by methods known in the art. Many suitable hydrophilic polymers and residues thereof can form hydrogels.

The molecular weight of the hydrophilic polymer or residue thereof can vary and will depend upon the selection of the hydrophilic polymer and/or the crosslinker and the particular application (e.g., whether the hydrogel is to be used to coat a support). In one example, the hydrophilic polymer can have a molecular weight of from about 2,000 Da to about 2,000,000 Da. In another aspect, the molecular weight of the hydrophilic polymer is about 5,000; 10,000; 20,000; 30,000; 40,000; 50,000; 75,000; 100,000; 200,000; 250,000; 300,000; 350,000; 400,000; 450,000; 500,000; 550,000; 600,000; 650,000; 700,000; 750,000; 800,000; 850,000; 900,000; 950,000; 1,000,000; 1,500,000; or 2,000,000 Da, where any stated values can form a lower and/or upper endpoint of a molecular weight range as appropriate.

Suitable hydrophilic polymers and residues thereof can include any number of polymers based on diol- or glycol-containing linkages, for example, polymers comprising polyethylene glycol (PEG), also known as polyethylene oxide (PEO), and polypropylene oxide (PPO). Other suitable examples include polymers comprising multiple segments or blocks of PEG alternating with blocks of polyester, for example, POLYACTIVE™ is a copolymer that has large blocks of PEG alternating with blocks of poly(butylene terephthalate).

In one example, the hydrophilic polymer or residue thereof comprises a multi-branched polymer (e.g., multi-armed PEG). Multi-branched polymers are polymers that have various polymeric chains (termed “arms” or “branches”) that radiate out from a central core. For example, the hydrophilic polymer or residue thereof can comprise a 2, 3, 4, 5, 6, 7, 8, 9, or 10 armed-PEGs. Such multi-arm polymers are commercially available or can be synthesized by methods known in the art.

Many suitable multi-armed polymers are referred to as dendrimers. The term “dendrimer” means a branched polymer that possesses multiple generations, where each generation creates multiple branch points. “Dendrimers” can include dendrimers having defects in the branching structure, dendrimers having an incomplete degree of branching, crosslinked and uncrosslinked dendrimers, asymmetrically branched dendrimers, star polymers, highly branched polymers, highly branched copolymers and/or block copolymers of highly branched and not highly branched polymers.

Any dendrimer can be used in the disclosed compositions and methods. Suitable examples of dendrimers that can be used include, but are not limited to, poly(propyleneimine) (DAB) dendrimers, benzyl ether dendrimers, phenylacetylene dendrimers, carbosilane dendrimers, convergent dendrimers, polyamine, and polyamide dendrimers. Other useful dendrimers include, for example, those described in U.S. Pat. Nos. 4,507,466, 4,558,120, 4,568,737 and 4,587,329, as well as those described in Dendritic Molecules, Concepts, Syntheses, Perspectives. Newkome, et al., VCH Publishers, Inc. New York, N.Y. (1996), which are incorporated by reference herein for at least their teachings of dendrimers.

In one example, the hydrophilic polymer or residue thereof comprises a triblock polymer of poly(ethylene oxide)-poly(propylene oxide)-poly(ethylene oxide). These polymers are referred to as PLUORONICS™. PLUORONICS™ are commercially available from BASF (Florham Park, N.J.) and have been used in numerous applications as emulsifiers and surfactants in foods, as well as gels and blockers of protein adsorption to hydrophobic surfaces in medical devices. These materials have low acute oral and dermal toxicity, and do not cause irritation to eyes or inflammation of internal tissues in man. The hydrophobic PPO block adsorbs to hydrophobic (e.g., polystyrene) surfaces, while the PEO blocks provide a hydrophilic coating that is protein-repellent. PLUORONICS™ have low toxicity and are approved by the FDA for direct use in medical applications and as food additives. Surface treatments with PLUORONICS™ can also reduce platelet adhesion, protein adsorption, and bacterial adhesion.

In another example, the hydrophilic polymer or residue thereof comprises a triblock polymer of poly(ethylene oxide)-poly(propylene oxide)-poly(ethylene oxide), wherein the polymer has a molecular weight of from 1,000 Da to 100,000 Da. In still another example, the hydrophilic polymer or residue thereof is a triblock polymer of poly(ethylene oxide)-poly(propylene oxide)-poly(ethylene oxide), wherein the polymer has a molecular weight of from having a lower endpoint of 1,000 Da, 2,000 Da, 3,000 Da, 5,000 Da, 10,000 Da, 15,000 Da, 20,000 Da, 30,000 and an upper endpoint of 5,000 Da, 10,000 Da, 15,000 Da, 20,000 Da, 25,000 Da, 30,000 Da, 40,000 Da, 50,000 Da, 60,000 Da, 70,000 Da, 80,000 Da, 90,000 Da, or 100,000 Da, wherein any lower endpoint can be matched with any upper endpoint, wherein the lower endpoint is less than the upper endpoint. In a further example, the hydrophilic polymer or residue thereof comprises a triblock polymer of poly(ethylene oxide)-poly(propylene oxide)-poly(ethylene oxide), wherein the polymer has a molecular weight of from 5,000 Da to 15,000 Da. In yet a further example, the triblock polymer of poly(ethylene oxide)-poly(propylene oxide)-poly(ethylene oxide) is PEO103-PPO39-PEO103, PEO132-PPO50-PEO132, or PEO100-PPO65-PEO100. In yet another example, the polymer is PEO103-PPO39-PEO103, PEO132-PPO50-PEO132, or PEO100-PPO65-PEO100.

Additional hydrophilic polymers and residues thereof can be those based on acrylic acid derivatives, such homopolymers or copolymers of as poly(meth)acrylate, polyvinyl alcohol, polyacrylonitrile, polyacrylamides, poly(alkylcyanoacrylates), and the like. Still other examples include polymers based on organic acids such as, but not limited to, polyglucuronic acid, polyaspartic acid, polytartaric acid, polyglutamic acid, polyfumaric acid, polylactide, and polyglycolide, including copolymers thereof. For example, polymers can be made from lactide and/or glycolide monomer units along with a polyether hydrophilic core segment as a single block in the backbone of the polymer. Suitable hydrophilic polymers that are based on esters include, but are not limited to, poly(ortho esters), poly(block-ether esters), poly(ester amides), poly(ester urethanes), polyphosphonate esters, polyphosphoesters, polyanhydrides, and polyphosphazenes, including copolymers thereof.

Still further examples of hydrophilic polymers and residues thereof include, but are not limited to, polyhydroxyalkanoates, poly(propylene fumarate), polyvinylpyrrolidone, polyvinyl polypyrrolidone, polyvinyl N-methylpyrrolidone, hydroxypropylcellulose, methylcellulose, sodium alginate, gelatin, acid-hydrolytically-degraded gelatin, agarose, carboxymethylcellulose, carboxypolymethylene, poly(hydroxypropyl methacrylate), poly(hydroxyethyl methacrylate), and poly(2-hydroxypropyl methacrylamide).

Hydrophilic polymers or residues thereof that are particularly suitable are those that form hydrogels. Examples of hydrogels useful herein include, but are not limited to, aminodextran, dextran, DEAE-dextran, chondroitin sulfate, dermatan, heparan, heparin, chitosan, polyethyleneimine, polylysine, dermatan sulfate, heparan sulfate, alginic acid, pectin, carboxymethylcellulose, hyaluronic acid, agarose, carrageenan, starch, polyvinyl alcohol, cellulose, polyacrylic acid, polyacrylamide, polyethylene glycol, or the salt or ester thereof, or a mixture thereof. In one example, the hydrogel can comprise carboxymethyl dextran having a molecular weight of from 5,000 Da to 100,000 Da, 5,000 Da to 90,000 Da; 10,000 Da to 90,000 Da; 20,000 Da to 90,000 Da; 30,000 Da to 90,000 Da; 40,000 Da to 90,000 Da; 50,000 Da to 90,000 Da; or 60,000 Da to 90,000 Da. Still other examples of hydrogels include, but are not limited to, poly(N-isopropyl acrylamide), poly(hydroxy ethylmethacrylate), poly(vinyl alcohol), poly(acrylic acid), polyethylene glycol diacrylate, polyethylene glycol dimethacrylate, and combinations thereof.

In further examples, the hydrophilic polymer or residue thereof can be a polysaccharide. Any polysaccharide known in the art can be used herein. Examples of polysaccharides include starch, cellulose, glycogen or carboxylated polysaccharides such as alginic acid, pectin, carboxymethyl amylose, or carboxymethylcellulose. Further, any of the polyanionic polysaccharides disclosed in U.S. Pat. No. 6,521,223, which is incorporated by reference in its entirety, can be used as the hydrophilic polymer or residue thereof. In one aspect, the polysaccharide is a glycosaminoglycan (GAG). A GAG is one molecule with many alternating subunits. For example, hyaluronan is (GlcNAc-GlcUA-)ₓ. Other GAGs are sulfated at different sugars. Generically, GAGs are represented by the formula A-B-A-B-A-B, where A is an uronic acid and B is an aminosugar that is either O- or N-sulfated, where the A and B units can be heterogeneous with respect to epimeric content or sulfation.

There are many different types of GAGs, having commonly understood structures, which, for example, are within the disclosed compositions, such as chondroitin, chondroitin sulfate, dermatan, dermatan sulfate, heparin, or heparan sulfate. Any GAG known in the art can be used in any of the methods described herein. Glycosaminoglycans can be purchased from Sigma, and many other biochemical suppliers. Alginic acid, pectin, and carboxymethylcellulose are among other carboxylic acid containing polysaccharides useful in the methods described herein.

In one example, the polysaccharide is hyaluronan (HA). HA is a non-sulfated GAG. Hyaluronan is a well known, naturally occurring, water soluble polysaccharide composed of two alternatively linked sugars, D-glucuronic acid and N-acetylglucosamine. The polymer is hydrophilic and highly viscous in aqueous solution at relatively low solute concentrations. It often occurs naturally as the sodium salt, sodium hyaluronate. Other salts such as potassium hyaluronate, magnesium hyaluronate, and calcium hyaluronate, are also suitable. Methods of preparing commercially available hyaluronan and salts thereof are well known. Hyaluronan can be purchased from Seikagaku Company, Clear Solutions Biotech, Inc., Pharmacia Inc., Sigma Inc., and many other suppliers. For high molecular weight hyaluronan it is often in the range of about 100 to about 10,000 disaccharide units. In another aspect, the lower limit of the molecular weight of the hyaluronan is from about 1,000 Da, 2,000 Da, 3,000 Da, 4,000 Da, 5,000 Da, 6,000 Da, 7,000 Da, 8,000 Da, 9,000 Da, 10,000 Da, 20,000 Da, 30,000 Da, 40,000 Da, 50,000 Da, 60,000 Da, 70,000 Da, 80,000 Da, 90,000 Da, or 100,000 Da, and the upper limit is 200,000 Da, 300,000 Da, 400,000 Da, 500,000 Da, 600,000 Da, 700,000 Da, 800,000 Da, 900,000 Da, 1,000,000 Da, 2,000,000 Da, 4,000,000 Da, 6,000,000 Da, 8,000,000 Da, or 10,000,000 Da where any of the lower limits can be combined with any of the upper limits.

It is also contemplated that the hydrophilic polymer can have hydrolysable or biochemically cleavable groups incorporated into the polymer network structure. Examples of such hydrogels are described in U.S. Pat. Nos. 5,626,863, 5,844,016, 6,051,248, 6,153,211, 6,201,065, 6,201,072, all of which are incorporated herein by reference in their entireties.

As noted previously, the disclosed hydrophilic polymers, R′, can contain at least one cycloaddition reactive moiety, X, as are described herein. In other examples, the hydrophilic polymer can comprise 1, 2, 3, 4, 5, 6, 7, 8, 9, or 10 cycloaddition reactive moieties. In still other examples, the hydrophilic polymer can comprise greater than or equal to 10, 15, or 20 cycloaddition reactive moieties. When the disclosed hydrophilic polymers comprise more than one cycloaddition reactive moieties, the reactive moieties can be the same or different. The number of cycloaddition reactive moieties present on the hydrophilic polymer can vary depending upon the amounts of type of hydrophilic polymer, the type of crosslinker, the type of cycloaddition reactive moieties, preference, and the like.

The cycloaddition reactive moieties can be produced in various ways depending on the particular hydrophilic polymer and the particular cycloaddition reactive moiety. For example, monomer containing a particular cycloaddition moiety can be polymerized together to form a hydrophilic polymer or a segment of the hydrophilic polymer. Also, a functional group on a hydrophilic polymer can be converted chemically to a cycloaddition reactive moiety. For example, hydroxyl groups on a polymer can be esterified with an azide containing acid. The result is a polymer functionalized with an azide, one of the cycloaddition reactive groups disclosed herein.

Crosslinker and Residue Thereof

The crosslinker, L′, can be any compound that contains at least two cycloaddition reactive moieties, as are described herein. For example, the crosslinker can comprise 2, 3, 4, 5, 6, 7, 8, 9, or 10 cycloaddition reactive moieties. In other examples, the crosslinker or residue thereof can comprise greater than or equal to 10, 15, or 20 cycloaddition reactive moieties. The cycloaddition reactive moieties can be the same or different. The number of cycloaddition reactive moieties, Y, present on the crosslinker can vary depending upon the amounts of type of hydrophilic polymer, the type of crosslinker, the type of cycloaddition reactive moieties, preference, and the like.

The crosslinker or residue thereof need not be hydrophilic, although in many cases it can be hydrophilic and contain one or more hydrophilic segments. When the crosslinker comprises a hydrophilic polymer or segment thereof, any of the hydrophilic polymers and segments thereof disclosed herein can be used.

In some example, the crosslinker or residue thereof can comprise a C₁-C₆ branched or straight-chain alkyl, such as methyl, ethyl, n-propyl, isopropyl, n-butyl, isobutyl, sec-butyl, tert-butyl, n-pentyl, isopentyl, neopentyl, sec-pentyl, or hexyl. In a specific example, the crosslinker or residue thereof can comprise a polyalkylene (i.e., —(CH₂)ₙ—, wherein n is from 1 to 5, from 1 to 4, from 1 to 3, or from 1 to 2). In another example, the crosslinker or residue thereof can comprise a C₁-C₆ branched or straight-chain alkoxy such as a methoxy, ethoxy, n-propoxy, isopropoxy, n-butoxy, isobutoxy, sec-butoxy, tert-butoxy, n-pentoxy, isopentoxy, neopentoxy, sec-pentoxy, or hexoxy.

In still other examples, the crosslinker or residue thereof can comprise a C₂-C₆ branched or straight-chain alkyl, wherein one or more of the carbon atoms are substituted with oxygen (e.g., an ether) or an amino group. For example, a suitable crosslinker or residue thereof can include, but is not limited to, a methoxymethyl, methoxyethyl, methoxypropyl, methoxybutyl, ethoxymethyl, ethoxyethyl, ethoxypropyl, propoxymethyl, propoxyethyl, methylaminomethyl, methylaminoethyl, methylaminopropyl, methylaminobutyl, ethylaminomethyl, ethylaminoethyl, ethylaminopropyl, propylaminomethyl, propylaminoethyl, methoxymethoxymethyl, ethoxymethoxymethyl, methoxyethoxymethyl, methoxymethoxyethyl, and the like, and derivatives thereof. In one specific example, the crosslinker or residue thereof can comprise a methoxymethyl (i.e., —CH₂—O—CH₂—). In another specific example, the crosslinker or residue thereof can comprise a polyether (e.g., —(OCH₂CH₂)ₘ—, wherein m is an integer from 2 to 10 (i.e., 2, 3, 4, 5, 6, 7, 8, 9, or 10).

The reaction between the crosslinker and the hydrophilic polymer results in a chemical bond that links the crosslinker to the hydrophilic polymer, i.e., Z in Formula I. As noted herein, such reactions can occur as a result of a cycloaddition reaction (e.g., a 3+2 or 2+2 cycloaddition) between the cycloaddition reactive moieties on the hydrophilic polymer and crosslinker.

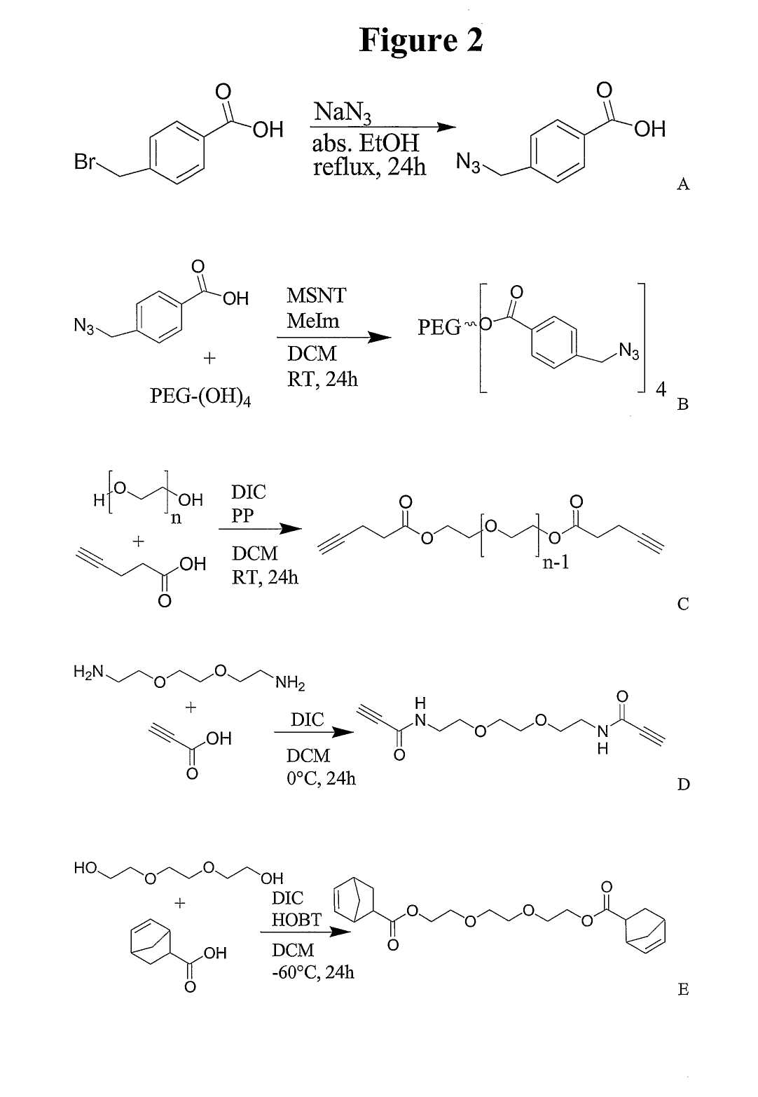

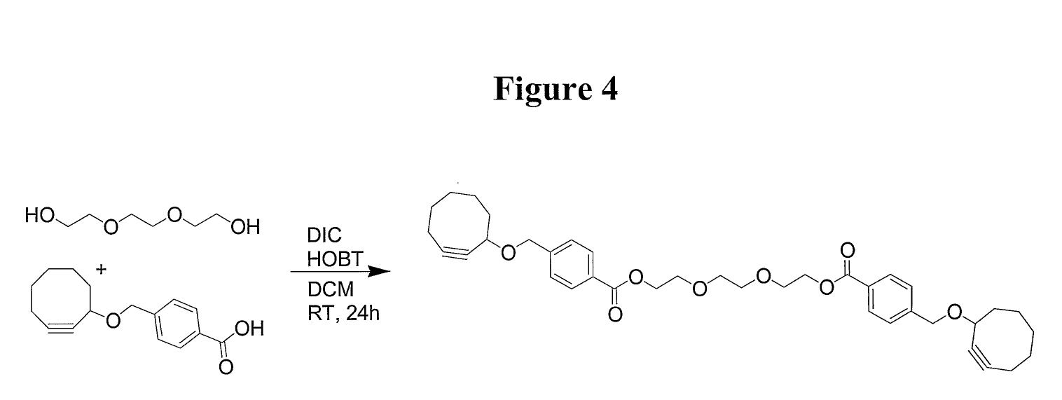

Suitable crosslinkers or residues thereof can be obtained from commercial sources or can be prepared by methods known in the art. For example, α,β-unsaturated acids can be coupled to crosslinkers that contain hydroxyl or amide groups using well known coupling methods (e.g., DIC or DCC couplings). FIG. 2, Schemes C-E, and FIG. 4 illustrate the synthetic routes for several suitable crosslinkers.

Cycloaddition Reactive Moiety

The hydrophilic polymer and the crosslinker both contain cycloaddition reactive moiety. These moieties are denoted X and Y in Scheme 1. A cycloaddition reactive moiety is any chemical functionality that can undergo a 3+2 or 2+2 cycloaddition reaction. The cycloaddition reactive moiety on the hydrophilic polymer, denoted X, reacts with the cycloaddition reactive moiety on the crosslinker, denoted Y, to form a covalent link, Z, between the remaining residues of the hydrophilic polymer and the crosslinker (i.e., R and L, respectively in Formula I).

The type of cycloaddition reactive moieties used will depend on the particular cycloaddition reaction. For example, if the cycloaddition reaction is a 3+2 cycloaddition reaction, then the cycloaddition reactive moieties can be a 1,3-dipolar group and a dipolarophile as disclosed herein. If the cycloaddition reaction is a 2+2 cycloaddition reaction, then the cycloaddition reactive moieties can be photoreactive sites.

3+2 Cycloaddition

A 3+2 cycloaddition involves the reaction of a compound having a 1,3-dipolar group with a dipolarophile. A general reaction scheme that shows the reaction between a 1,3-dipolar group (shown as A¹=A²-A³) and a dipolarophile (shown as A⁴=A⁵) is depicted in Scheme 2.

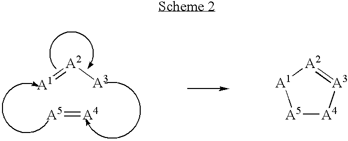

The resulting product of a 3+2 cycloaddition is typically a 5 membered ring structure.

In many examples disclosed herein, the cycloaddition reactive moieties can be a 1,3-dipolar group and a dipolarophile. The 1,3-dipolar group can, in some examples, be present on the hydrophilic polymer and the dipolarophile can be present on the crosslinker. That is, referring to Scheme 1, X can be a 1,3-dipolar group and Y can be a dipolarophile. Alternatively, the 1,3-dipolar group can be present on the crosslinker and the dipolarophile can be present on the hydrophilic polymer (e.g. Y can be a 1,3-dipolar group and X can be a dipolarophile in Scheme 1). In still other examples, the hydrophilic polymer can comprise a 1,3-dipolar group and a dipolarophile (e.g., more than one X is present on R′ and some are a 1,3-dipolar group and others are dipolarophiles) and the crosslinker can also comprise a 1,3-dipolar group and a dipolarophile (e.g., some Y groups are 1,3-dipolar groups and some are dipolarophiles). It is also possible that the same or different 1,3-dipolar groups be present on the hydrophilic polymer and/or the crosslinker. For example, more than one type of 1,3-dipolar group can be present on the hydrophilic polymer and/or the crosslinker. In another example, more than one type of dipolarophile can be present on the hydrophilic polymer and/or crosslinker. Further, the same or different dipolarophiles can be present on the hydrophilic polymer and/or crosslinker.

The term “1,3-dipolar group” as used herein is any group that can react with a dipolarophile, as described herein. A 1,3-dipolar group is group whereby oppositely charged dipoles can be shown through resonance as being distributed over three atoms. Examples of suitable 1,3-dipolar groups include, but are not limited to, those shown in Table 1.

TABLE 1
Exemplary 1,3-dipole groups

| | |
|---|---|
| 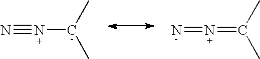 | Diazoalkanes |
| 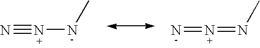 | Azides |
| 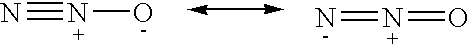 | Nitrous oxide |
| 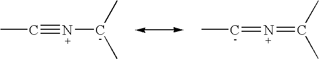 | Nitrile ylides |
| 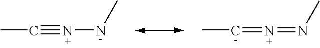 | Nitrile imines |
| 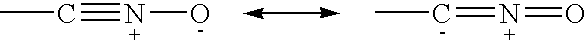 | Nitrile oxides |
| 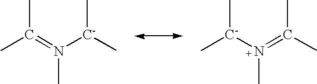 | Azomethine ylides |
| 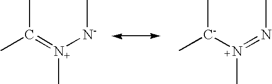 | Azomethine imines |
| 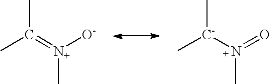 | Nitrones |
| 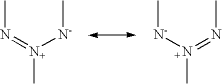 | Azimines |
| 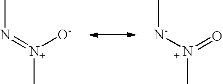 | Azoxy groups |
| 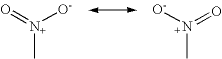 | Nitro groups |
| 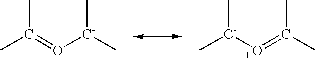 | Carbonyl ylides |
| 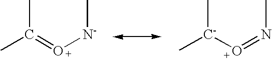 | Carbonyl imines |
| 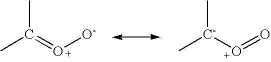 | Carbonyl oxides |
| 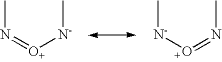 | Nitrosimines |
| 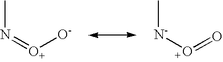 | Nitrosoxides |
| 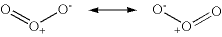 | Ozone |

The term “dipolarophile” as used herein is any group that can react with a 1,3-dipolar group. Examples of suitable dipolarophiles are substituted or unsubstituted alkene, cycloalkene, alkyne, cycloalkyne, or aryl groups. In some examples, the dipolarophile can be an electron deficient dipolarophile. The term “electron-deficient dipolarophile” as used herein is a dipolarophile group where a π-electron system (e.g., carbon-carbon or carbon-heteroatom double or triple bond) is attached to an electron-withdrawing group or is part of a strained ring system. Examples of electron-withdrawing groups include, but are not limited to, a nitro group, a cyano group, an ester group, an aldehyde group, a keto group, a sulfo-oxo group, or an amide group. Examples of electron deficient dipolarophile groups where the dipolarophile is part of a strained ring system include, but are not limited to, a cyclopentene, cyclohexene, cyclohexadiene, cyclooctyne, norbornene, and the like.

As shown in Scheme 2, the product of a 3+2 cycloaddition is a 5 membered ring. Accordingly, when the hydrophilic polymer and crosslinker react, the moiety connecting the remaining hydrophilic residue to the crosslinker residue can be a 5 membered ring. Referring to Formula I, Z can be the 5 membered ring produced by the cycloaddition reaction between the 1,3-dipolar group and dipolarophile. Examples of Z are shown in Table 2.

TABLE 2
Exemplary moieties formed by 3 + 2 cycloaddition

| | | |
|---|---|---|
| Azide 1,3-dipolar group Alkene dipolarophile | 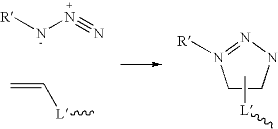 | Triazoline |
| Azide 1,3-dipolar group Alkyne dipolarophile | 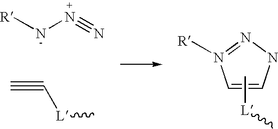 | Triazole |
| Azide 1,3-dipolar group Norbornenyl dipolarophile | 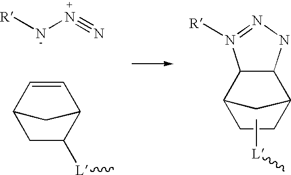 | |

2+2 Cycloaddition

In the disclosed compositions and methods, the hydrophilic polymer and the crosslinker can be coupled together by a 2+2 cycloaddition reaction. That is, the cycloaddition reactive moieties on the hydrophilic polymer and the crosslinker can undergo a 2+2 cycloaddition. A 2+2 cycloaddition is a light-induced reaction between two photoreactive sites, at least one of which is electronically excited. Specifically, the 2+2 cycloaddition involves addition of a 2π-component of a first double bond to a 2π-component of a second double bond, as shown in Scheme 3. Alternatively, the reaction can proceed by way of a 2π-component of triple bonds. The result is that two carbon-carbon bonds or a carbon-carbon and a carbon-heteroatom single bond are formed in a single step to produce a 4 membered cyclic structure. Generally, 2+2 cycloaddition reactions can proceed with high efficiency and a high degree of stereospecificity and regiospecificity.

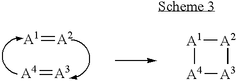

Under the rules of orbital symmetry, such 2+2 cycloadditions are thermally forbidden, but photochemically allowed.

Suitable 2+2 cycloaddition reactive moieties for used in the disclosed compositions and methods include moieties capable of undergoing 2+2 cycloaddition to form a ring structure when exposed to light of an appropriate wavelength. Specific examples include, but are not limited to, alkenes (e.g., vinyl groups and acrylates), alkynes, carbonyl containing groups (e.g., ketones, aldehydes, esters, carboxylic acids), and imines. A detailed discussion of suitable 2+2 cycloaddition reactive moieties can be found in Guillet, Polymer Photophysics and Photochemistry, Ch. 12 (Cambridge University Press: Cambridge, London). Generally, double bonds that are not part of a highly conjugated system (e.g. benzene will not work) are suitable. Sterically-hindered, electron deficient double bonds, such as found in maleimide, are also suitable. The disclosed hydrophilic polymers can comprise the same or different 2+2 cycloaddition reactive moieties. Similarly, the disclosed crosslinkers can comprise the same or different 2+2 cycloaddition moieties.

In some examples, a 2+2 cycloaddition between two carbon-carbon double bonds (e.g., one on the hydrophilic polymer and one the crosslinker) forms cyclobutanes and those between alkenes and carbonyl groups form oxetanes. Cycloadditions between two alkenes to form cyclobutanes can be carried out by photo-sensitization with mercury or directly with short wavelength light, as described in Yamazaki et al., J. Am. Chem. Soc. 1969, 91, 520. The reaction works particularly well with electron-deficient double bonds because electron-poor olefins are less likely to undergo undesirable side reactions. Cycloadditions between carbon-carbon and carbon-oxygen double bonds, such as α,β-unsaturated ketones, form oxetanes (Weeden, In Synthetic Organic Photochemistry, Chapter 2, W. M. Hoorspool (ed.) Plenum, New York, 1984) and enone addition to alkynes (Cargill et al., J. Org. Chem. 1971, 36, 1423).

Some specific 2+2 cycloaddition reactive moieties include, but are not limited to, dialkyl maleimides, maleimide/N-hydroxysuccinimide (NHS) ester derivatives such as 3-maleimidoproprionic acid hydroxysuccinimide ester, 3-maleimidobenzoic acid N-hydroxy succinimide, N-succinimidyl 4-malimidobutyrate, N-succinimidyl 6-maleimidocaproate, N-succinimidyl 8-maleimidocaprylate, and N-succinimidyl 11-maleimidoundecaoate, vinyl derivatives and acylated derivatives.

### Specific Examples

In some specific examples of the polymer compositions disclosed herein, the hydrophilic polymer can be a multi-branched or graft polymer comprising one or more cycloaddition reactive moieties. Multi-branched polymers, such as multi-arm PEG, include those polymers which have polymeric units comprising each arm. Graft polymers, such as poly(hydroxypropyl methacrylate) and poly(hydroxyethyl methacrylate), include those polymers which have polymeric units comprising either a linear chain or multiple branches as well as monomeric units comprising multiple branches.

In other examples of the disclosed polymer compositions, the hydrophilic polymer can be a multi-armed PEG polymer comprising one or more cycloaddition reactive moieties. Specifically, the hydrophilic polymer can comprise a multi-arm PEG polymer comprising one or more 1,3-dipolar groups and/or dipolarophiles. Also, the crosslinker can be a multi-arm PEG polymer comprising one or more 1,3-dipolar groups and/or dipolarophiles. In further specific examples, the hydrophilic polymer can comprise one or more azide group. In still other examples, the crosslinker can comprises one or more alkyne groups.

In some examples, Z can be a triazole or a triazoline group. In other examples, Z can be a cyclobutyl group.

In other specific examples of the polymer compositions disclosed herein, the hydrophilic polymer can be a graft copolymer or homopolymer, such as poly(hydroxypropyl methacrylate), poly(hydroxyethyl methacrylate), poly(2-hydroxypropyl methacrylamide), on which grafts comprise one or more cycloaddition reactive moieties. Specifically, the hydrophilic polymer can comprise a graft copolymer or homopolymer, such as poly(hydroxypropyl methacrylate), poly(hydroxyethyl methacrylate), poly(2-hydroxypropyl methacrylamide), comprising one or more 1,3-dipolar groups and/or dipolarophiles. Also, the crosslinker can be a graft copolymer or homopolymer, such as poly(hydroxypropyl methacrylate), poly(hydroxyethyl methacrylate), or poly(2-hydroxypropyl methacrylamide) comprising one or more 1,3-dipolar groups and/or dipolarophiles. In further specific examples, the hydrophilic polymer can comprise one or more azide group. In still other examples, the crosslinker can comprises one or more alkyne groups.

Pharmaceutically Acceptable Salts

Any of the polymeric compositions and components thereof described herein can be a pharmaceutically acceptable salt or ester thereof if they possess groups that are capable of being converted to a salt or ester. Pharmaceutically acceptable salts are prepared by treating the free acid with an appropriate amount of a pharmaceutically acceptable base. Representative pharmaceutically acceptable bases are ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, histidine, and the like.

In some examples, if the polymeric composition or component thereof possesses a basic group, it can be protonated with an acid such as, for example, HCl or H₂SO₄, to produce the cationic salt. In one example, the compound can be protonated with tartaric acid or acetic acid to produce the tartarate or acetate salt, respectively. In another example, the reaction of the compound with the acid or base is conducted in water, alone or in combination with an inert, water-miscible organic solvent, at a temperature of from about 0° C. to about 100° C., such as at room temperature. In certain situations, where applicable, the molar ratio of the disclosed compounds to base is chosen to provide the ratio desired for any particular salts.

Ester derivatives are typically prepared as precursors to the acid form of the compounds and accordingly can serve as prodrugs. Generally, these derivatives will be lower alkyl esters such as methyl, ethyl, and the like.

Pharmaceutical Polymeric Compositions

In some examples, any of the compositions and components produced by the methods described herein can include at least one bioactive agent that attached (either covalently or non-covalently) to the polymeric composition. The resulting pharmaceutical polymeric composition can provide a system for sustained, continuous delivery of drugs and other biologically-active agents to tissues adjacent to or distant from the application site. The bioactive agent is capable of providing a local or systemic biological, physiological, or therapeutic effect in the biological system to which it is applied. For example, the bioactive agent can act to control infection or inflammation, enhance cell growth and tissue regeneration, control tumor growth, act as an analgesic, promote anti-cell attachment, and enhance bone growth, among other functions. Other suitable bioactive agents can include anti-viral agents, hormones, antibodies, or therapeutic proteins. Other bioactive agents include prodrugs, which are agents that are not biologically active when administered but, upon administration to a subject are converted to bioactive agents through metabolism or some other mechanism. Additionally, any of the compositions disclosed herein can contain combinations of two or more bioactive agents.

In some examples, the bioactive agents can include substances capable of preventing an infection systemically in the biological system or locally at the defect site, as for example, anti-inflammatory agents such as, but not limited to, pilocarpine, hydrocortisone, prednisolone, cortisone, diclofenac sodium, indomethacin, 6∝-methyl-prednisolone, corticosterone, dexamethasone, prednisone, and the like; antibacterial agents including, but not limited to, penicillin, cephalosporins, bacitracin, tetracycline, doxycycline, gentamycin, chloroquine, vidarabine, and the like; analgesic agents including, but not limited to, salicylic acid, acetaminophen, ibuprofen, naproxen, piroxicam, flurbiprofen, morphine, and the like; local anesthetics including, but not limited to, cocaine, lidocaine, benzocaine, and the like; immunogens (vaccines) for stimulating antibodies against hepatitis, influenza, measles, rubella, tetanus, polio, rabies, and the like; peptides including, but not limited to, leuprolide acetate (an LH-RH agonist), nafarelin, and the like. All of these agents are commercially available from suppliers such as Sigma Chemical Co. (Milwaukee, Wis.).

Additionally, a substance or metabolic precursor which is capable of promoting growth and survival of cells and tissues or augmenting the functioning of cells is useful, as for example, a nerve growth promoting substance such as a ganglioside, a nerve growth factor, and the like; a hard or soft tissue growth promoting agent such as fibronectin (FN), human growth hormone (HGH), a colony stimulating factor, bone morphogenic protein, platelet-derived growth factor (PDGF), insulin-derived growth factor (IGF-I, IGF-II), transforming growth factor-α (TGF-α), transforming growth factor-β (TGF-β), epidermal growth factor (EGF), fibroblast growth factor (FGF), interleukin-1 (IL-1), vascular endothelial growth factor (VEGF) and keratinocyte growth factor (KGF), dried bone material, and the like; and antineoplastic agents such as methotrexate, 5-fluorouracil, adriamycin, vinblastine, cisplatin, tumor-specific antibodies conjugated to toxins, tumor necrosis factor, and the like.

Other useful substances include hormones such as progesterone, testosterone, and follicle stimulating hormone (FSH) (birth control, fertility-enhancement), insulin, and the like; antihistamines such as diphenhydramine, and the like; cardiovascular agents such as papaverine, streptokinase and the like; anti-ulcer agents such as isopropamide iodide, and the like; bronchodilators such as metaproternal sulfate, aminophylline, and the like; vasodilators such as theophylline, niacin, minoxidil, and the like; central nervous system agents such as tranquilizer, B-adrenergic blocking agent, dopamine, and the like; antipsychotic agents such as risperidone, narcotic antagonists such as naltrexone, naloxone, buprenorphine; and other like substances. All of these agents are commercially available from suppliers such as Sigma Chemical Co. (Milwaukee, Wis.).

The pharmaceutical polymeric compositions can be prepared using techniques known in the art. In one aspect, the composition is prepared by admixing a polymeric composition disclosed herein with a bioactive agent. The term “admixing” is defined as mixing the two components together so that there is no chemical reaction or physical interaction. The term “admixing” also includes the chemical reaction or physical interaction between the compound and the pharmaceutically-acceptable compound. Covalent bonding to reactive therapeutic drugs, e.g., those having reactive carboxyl groups, can be undertaken on the compound. For example, first, carboxylate-containing chemicals such as anti-inflammatory drugs ibuprofen or hydrocortisone-hemisuccinate can be converted to the corresponding N-hydroxysuccinimide (NHS) active esters and can further react with the OH group of a hydrophilic polymer. Second, non-covalent entrapment of a bioactive agent in any of the disclosed compositions is also possible. Third, electrostatic or hydrophobic interactions can facilitate retention of a bioactive agent in the disclosed compositions. Fourth, a free cycloaddition reactive moiety in the composition can react with a cycloaddition reactive moiety (e.g., alkene or alkyne) in a bioactive agent.

It will be appreciated that the actual preferred amounts of bioactive agent in a specified case will vary according to the specific compound being utilized, the particular compositions formulated, the mode of application, and the particular situs and subject being treated. Dosages for a given host can be determined using conventional considerations, e.g., by customary comparison of the differential activities of the subject compounds and of a known agent, e.g., by means of an appropriate conventional pharmacological protocol. Physicians and formulators skilled in the art of determining doses of pharmaceutical compounds will have no problems determining dose according to standard recommendations (Physicians Desk Reference, Barnhart Publishing (1999)).

Pharmaceutical polymeric compositions described herein can be formulated in any excipient the biological system or entity can tolerate. Examples of such excipients include, but are not limited to, water, saline, Ringer's solution, dextrose solution, Hank's solution, and other aqueous physiologically balanced salt solutions. Nonaqueous vehicles, such as fixed oils, vegetable oils such as olive oil and sesame oil, triglycerides, propylene glycol, polyethylene glycol, and injectable organic esters such as ethyl oleate can also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosol, cresols, formalin, and benzyl alcohol.

Pharmaceutical carriers are known to those skilled in the art. These most typically would be standard carriers for administration to humans, including solutions such as sterile water, saline, and buffered solutions at physiological pH.

Molecules intended for pharmaceutical delivery can be formulated in a pharmaceutical composition. Pharmaceutical compositions can include carriers, thickeners, diluents, buffers, preservatives, surface active agents and the like in addition to the molecule of choice. Pharmaceutical compositions can also include one or more active ingredients such as antimicrobial agents, anti-inflammatory agents, anesthetics, and the like.

The pharmaceutical polymeric composition can be administered in a number of ways depending on whether local or systemic treatment is desired, and on the area to be treated. Administration can be topically (including ophthalmically, vaginally, rectally, intranasally).

Preparations for administration include sterile aqueous or non-aqueous solutions, suspensions, and emulsions. Examples of non-aqueous carriers include water, alcoholic/aqueous solutions, emulsions or suspensions, including saline and buffered media. Parenteral vehicles, if needed for collateral use of the disclosed compositions and methods, include sodium chloride solution, Ringer's dextrose, dextrose and sodium chloride, lactated Ringer's, or fixed oils. Intravenous vehicles, if needed for collateral use of the disclosed compositions and methods, include fluid and nutrient replenishers, electrolyte replenishers (such as those based on Ringer's dextrose), and the like. Preservatives and other additives can also be present such as, for example, antimicrobials, anti-oxidants, chelating agents, and inert gases, and the like.

Formulations for topical administration can include ointments, lotions, creams, gels, drops, suppositories, sprays, liquids and powders. Conventional pharmaceutical carriers, aqueous, powder or oily bases, thickeners and the like can be necessary or desirable.

Dosing is dependent on severity and responsiveness of the condition to be treated, but will normally be one or more doses per day, with course of treatment lasting from several days to several months or until one of ordinary skill in the art determines the delivery should cease. Persons of ordinary skill can easily determine optimum dosages, dosing methodologies and repetition rates.

In one aspect, any of the disclosed compositions can include living cells. Examples of living cells include, but are not limited to, fibroblasts, hepatocytes, chondrocytes, stem cells, bone marrow, muscle cells, cardiac myocytes, neuronal cells, or pancreatic islet cells.

### Methods of Making

Disclosed herein are methods of making the disclosed polymeric compositions. These methods can also be used for crosslinking any of the components described herein to produce a polymeric composition. In one example, disclosed is a method of making a polymeric composition, comprising contacting a hydrophilic polymer comprising one or more cycloaddition reactive moieties with a crosslinker comprising two or more cycloaddition reactive moieties, wherein the cycloaddition reactive moieties undergo a cycloaddition reaction to provide the polymeric composition. In one example, the polymeric composition is not a polyacrylamide crosslinked with a photoactivated 2+2 cycloaddition reaction. The cycloaddition conditions can be conditions that result in a 3+2 cycloaddition reaction between the cycloaddition reactive moieties or a 2+2 cycloaddition reaction between the cycloaddition reactive moieties. In the disclosed methods, a cycloaddition reaction takes place between the cycloaddition reactive moiety on the hydrophilic polymer and the cycloaddition moieties on the crosslinker to result in a covalent attachment between the remaining hydrophilic polymer residue and crosslinker residue.

In some examples, the cycloaddition crosslinking that occurs in the disclosed methods can be based on click chemistry. The term “click chemistry” refers to any crosslinking chemistry that is highly favorable under mild conditions and was first coined by Valerie Fokin and K. Barry Sharpless in regards to the triazole-forming reaction between an azide and an alkyne in aqueous environment (Rostovtsev et al., Angew. Chem. Int. Ed. 2002, 41, 2596-9). This crosslinking chemistry, which has been used in drug discovery (Lee et al., J. Am. Chem. Soc. 2003, 125, 9588-9; Lewis et al., Angew. Chem. Int. Ed. 2002, 41, 1053-7; Lewis et al., J. Am. Chem. Soc. 2004, 126, 9152-3), fluorogenic probes (Zhou and Fahrni, J. Am. Chem. Soc. 2004, 126, 8862-3), and cell surface engineering (Link et al., J. Am. Chem. Soc. 2004, 126, 10598-602; Agard et al., J. Am. Chem. Soc. 2004, 126, 15046-7), typically requires the use of copper(I) as a catalyst that has known micromolar toxicity (Arciello et al, Biochem. Biophys. Res. Commun. 2005, 327, 454-9; Smet et al., Hum. Exp. Toxicol. 2003, 22, 89-93; Seth et al., Toxicol. In Vitro 2004, 18, 501-9). In order to reduce the risk of toxicity or inflammation, disclosed herein, in some examples, is the use of catalyst-free click chemistry, which can be accomplished using, for example, electron-deficient alkynes (Li et al., Tetrahedron Lett. 2004, 45, 3143-3146). All of the references disclosed in this paragraph are hereby incorporated by reference at least for their teaching of click chemistry.

In other examples, the cycloaddition conditions can be mild, at a pH of from about 0 to about 8, from about 1 to about 7, from about 2 to about 6, from about 3 to about 5, or from about 4 to about 8. In another example, the pH can be neutral or physiological pH. In another example, the cycloaddition reaction can occur in aqueous media or in biological fluids. For example, the composition or components thereof can be dissolved in water, which may also contain water-miscible solvents including, but not limited to, dimethylformamide, dimethylsulfoxide, and alcohols, diols, or glycerols. In other examples, the cycloaddition reaction can occur at from about minus 4° C. to about 90° C., from about 4° C. to about 80° C., from about 4° C. to about 70° C., from about 4° C. to about 60° C., from about 4° C. to about 50° C., from about 4° C. to about 40° C., from about 20° to about 40° C., or from about 25° C. to about 37° C. In another particular example, the cycloaddition reaction occurs at about 37° C. Further, the cycloaddition can occur in the presence of cells, biomolecules, tissues, and salts, such as are present in a biological system.

In one example, the cycloaddition reaction uses a catalyst. Suitable catalysts for 3+2 cycloadditions include copper salts (e.g., copper sulfate, copper bromide, and copper iodide) and other copper sources (e.g., copper wire). Catalyst may also be combined with reducing agents (e.g., sodium ascorbate, tris(carboxyethyl)phosphine) and/or stabilizing ligands (e.g., tris-triazolyl compounds). In other examples, the cycloaddition reactions are catalyst free. The uses of additional compounds that will facilitate crosslinking are also contemplated.

In the disclosed methods, any of the hydrophilic polymers and any of the crosslinkers disclosed herein can be used, including any of the cycloaddition reactive moieties disclosed herein.

Additional Crosslinking

It is also contemplated that the cycloaddition crosslinking disclosed herein can be used along with other crosslinking chemistries. For example, the disclosed polymeric compositions can contain crosslinking produce with other crosslinking chemistries before or after the cycloaddition based crosslinking.

For example, a polycarbonyl crosslinker can react with any of the hydrophilic polymers disclosed herein. The term “polycarbonyl crosslinker” is defined herein as a compound that possesses two or more groups represented by the formula A¹C(O)—, where A¹ is hydrogen, lower alkyl, or OA², where A² is a group that results in the formation of an activated ester. In one aspect, any of the hydrophilic polymers can be further crosslinked with a polyaldehyde. A polyaldehyde is a compound that has two or more aldehyde groups. In one aspect, the polyaldehyde is a dialdehyde compound. In one example, any compound possessing two or more aldehyde groups can be used as the polyaldehyde crosslinker. In another example, the polyaldehyde can be substituted or unsubstituted alkyl, alkenyl, alkynyl, cycloalkyl, cycloalkenyl, cycloalkynyl, ether, polyether, polyalkylene, ester, polyester, aryl, heteroaryl, and the like. In yet another example, the polyaldehyde can contain a polysaccharyl group or a polyether group. In a further aspect, the polyaldehyde can be a dendrimer or peptide. In one example, a polyether dialdehyde such as poly(ethylene glycol) propiondialdehyde (PEG) is useful in the compositions and methods described herein. PEG can be purchased from many commercial sources, such as Shearwater Polymers, Inc. (Huntsville, Ala.). In another example, the polyaldehyde is glutaraldehyde.

In another example, when the polycarbonyl compound is a polyaldehyde, the polyaldehyde can be prepared by the oxidation of terminal polyols or polyepoxides possessing two or more hydroxy or epoxy groups, respectively, using techniques known in the art.

The method of crosslinking generally involves reacting the hydrophilic polymer or polymeric composition with the polycarbonyl crosslinker in the presence of a solvent.

In one aspect, the reaction solvent is water. In addition, small amounts of water miscible organic solvents, such as an alcohol or DMF or DMSO, can be used as well. In one aspect, crosslinking can be performed at room temperature, for example, 25° C., but the crosslinking reaction can be performed within a range of temperatures from below about 4° C. to above about 90° C. but typically would be performed at from about 4° C. to about 60° C., more typically from about 4° C. to about 50° C., and more typically at about 4° C., or about, 30° C., or about 37° C. The reaction will also work at a variety of pHs, for example, pH from about 3 to about 10, or pH from about 4 to about 9, or pH from about 5 to about 8, or at neutral pH.

Functionalization of the Polymer Compositions

In addition to cycloaddition between the hydrophilic polymer and crosslinker, it can be desired that some of the cycloaddition reactive moieties not react so that they can be available for subsequent or orthogonal cycloaddition coupling reactions with other components, e.g., pharmaceutical compounds, markers, dyes, targeting moieties, DNA probes. Also contemplated herein are hydrophilic polymers and/or crosslinkers that contain a 3+2 cycloaddition reactive moiety and a 2+2 cycloaddition reactive moiety. In this way the disclosed polymer compositions can be crosslinked with one set of cycloaddition reactive moieties (e.g., a 1,3-dipolar group and a dipolarophile), leaving the other cycloaddition reactive moieties (e.g., photoreactive sites) free to undergo a 2+2 cycloaddition with another component. For example, during or after a 3+2 cycloaddition reaction to crosslink the disclosed polymeric compositions, additional 2+2 cycloaddition reactive moieties can be cyclized with various biomolecules. Alternatively, the 2+2 cycloaddition reactive moieties can be used to crosslink the polymer composition and additional 3+2 cycloaddition reactive moieties can be used to bind another component to the polymer composition. In a likewise fashion, the polymeric compositions can be attached to a solid support, such as glass or plastic, with 2+2 or 3+2 cycloaddition reactive moieties, whichever the case may be.

It is also contemplated that the polymer compositions can contain additional functionality other than cycloaddition reactive moieties, which can be used to couple other compounds to the polymeric compositions. For example, a bioactive agent can be linked to the polymeric composition through an ether, imidate, thioimidate, ester, amide, thioether, thioester, thioamide, carbamate, disulfide, hydrazide, hydrazone, oxime ether, oxime ester, or and amine linkage.

In some specific examples, a polymeric composition as disclosed herein can be modified with one or more different groups so that the composition forms a covalent bond with a bioactive agent or a solid support. In one example, if the bioactive agent or solid support has an amino group, it can react with one or more groups on the polymeric composition to form a covalent or non-covalent bond. For example, the amino group on the bioactive agent or support can react with a carboxymethyl-derivatized hydrogel such as carboxymethyl dextran to produce a new covalent bond.

In one example, the polymeric composition can be a hydrogel possessing one or more groups that can form covalent and/or non-covalent attachments to another component (e.g., a biomolecules or bioactive agent). For example, the hydrogel layer can comprise one or more cationic groups or one or more groups that can be converted to a cationic group. Examples of such groups include, but are not limited to, substituted or unsubstituted amino groups. In one example, when the hydrogel possesses cationic groups, the hydrogel can attach to components that possess negatively-charged groups to form electrostatic interactions. Conversely, the hydrogel can possess groups that can be converted to anionic groups (e.g., carboxylic acids or alcohols), wherein the hydrogel can electrostatically attach to positively-charged components. Also, the hydrogel can possess one or more groups capable of forming covalent bonds with the other component. Thus, it is contemplated that the hydrogel can form covalent and/or non-covalent bonds with the component.

Anti-Adhesion Polymeric Compositions

In some particular examples, the disclosed polymeric compositions can be further coupled to an anti-adhesion compound and/or a prohealing compound. The term “anti-adhesion compound” as referred to herein is defined as any compound that prevents cell attachment, cell spreading, cell growth, cell division, cell migration, or cell proliferation. In some examples, compounds that induce apoptosis, arrest the cell cycle, inhibit cell division, and stop cell motility can be used as the anti-adhesion compound. Examples of anti-adhesion compounds include, but are not limited to, anti-cancer drugs, anti-proliferative drugs, PKC inhibitors, ERK or MAPK inhibitors, cdc inhibitors, antimitotics such as colchicine or taxol, DNA intercalators such as adriamycin or camptothecin, or inhibitors of PI3 kinase such as wortmannin or LY294002. In one example, the anti-adhesion compound is a DNA-reactive compound such as mitomycin C. In another example, any of the oligonucleotides disclosed in U.S. Pat. No. 6,551,610, which is incorporated by reference in its entirety, can be used as the anti-adhesion compound. In another example, any of the anti-inflammatory drugs described below can be the anti-adhesion compound. Examples of anti-inflammatory compounds include, but are not limited to, methyl prednisone, low dose aspirin, medroxy progesterone acetate, and leuprolide acetate.

The formation of anti-adhesion polymeric compositions involves reacting the anti-adhesion compound with the polymer composition to form a new covalent bond. In one example, the anti-adhesion compound possesses a group that is capable of reacting with the polymeric composition (either through cycloaddition or through some other mechanism). The group present on the anti-adhesion compound that can react with the polymeric composition can be naturally-occurring or the anti-adhesion compound can be chemically modified to add such a group. In another example, the polymeric composition can be chemically modified so that it is more reactive with the anti-adhesion compound.

In some examples, the anti-adhesion polymeric composition can be formed by crosslinking the anti-adhesion compound with the polymeric composition. In one example, the anti-adhesion compound and the polymeric composition each possess at least one cycloaddition reactive moiety, which then can react with a crosslinker having at least two cycloaddition reactive moieties. Any of the cycloaddition reactive moieties described herein can be used in this respect. In one example, the crosslinker is a polyethylene glycol dialkyne.

The amount of the anti-adhesion compound relative the amount of the polymer composition can vary. In one example, the volume ratio of the anti-adhesion compound to the polymeric composition is from 99:1, 90:10, 80:20, 70:30, 60:40, 50:50, 40:60, 30:70, 20:80, 10:90, or 1:99. In one example, the anti-adhesion compound and the polymeric composition can react in air and are allowed to dry at room temperature. The resultant compound can then be rinsed with water to remove any unreacted anti-adhesion compound. The composite can optionally contain unreacted (i.e., free) anti-adhesion compound. The unreacted anti-adhesion compound can be the same or different anti-adhesion compound that is covalently bonded to the polymeric composition.

The anti-adhesion polymeric composition can also be composed of a prohealing compound. The term “prohealing compound” as defined herein is any compound that promotes cell growth, cell proliferation, cell migration, cell motility, cell adhesion, or cell differentiation. In one example, the prohealing compound includes a protein or synthetic polymer. Proteins useful in the methods described herein include, but are not limited to, an extracellular matrix protein, a chemically-modified extracellular matrix protein, or a partially hydrolyzed derivative of an extracellular matrix protein. The proteins can be naturally occurring or recombinant polypeptides possessing a cell interactive domain. The protein can also be mixtures of proteins, where one or more of the proteins are modified. Specific examples of proteins include, but are not limited to, collagen, elastin, decorin, laminin, or fibronectin.

In another example, the prohealing compound can be any of the supports disclosed in U.S. Pat. No. 6,548,081 B2, which is incorporated by reference in its entirety. In one example, the prohealing compound includes crosslinked alginates, gelatin, collagen, crosslinked collagen, collagen derivatives, such as, succinylated collagen or methylated collagen, cross-linked hyaluronan, chitosan, chitosan derivatives, such as, methylpyrrolidone-chitosan, cellulose and cellulose derivatives such as cellulose acetate or carboxymethyl cellulose, dextran derivatives such carboxymethyl dextran, starch and derivatives of starch such as hydroxyethyl starch, other glycosaminoglycans and their derivatives, other polyanionic polysaccharides or their derivatives, polylactic acid (PLA), polyglycolic acid (PGA), a copolymer of a polylactic acid and a polyglycolic acid (PLGA), lactides, glycolides, and other polyesters, polyoxanones and polyoxalates, copolymer of poly(bis(p-carboxyphenoxy)propane)anhydride (PCPP) and sebacic acid, poly(L-glutamic acid), poly(D-glutamic acid), polyacrylic acid, poly(DL-glutamic acid), poly(L-aspartic acid), poly(D-aspartic acid), poly(DL-aspartic acid), polyethylene glycol, copolymers of the above listed polyamino acids with polyethylene glycol, polypeptides, such as, collagen-like, silk-like, and silk-elastin-like proteins, polycaprolactone, poly(alkylene succinates), poly(hydroxy butyrate) (PHB), poly(butylene diglycolate), nylon-2/nylon-6-copolyamides, polydihydropyrans, polyphosphazenes, poly(ortho ester), poly(cyano acrylates), polyvinylpyrrolidone, polyvinylalcohol, poly casein, keratin, myosin, and fibrin. In another example, highly crosslinked HA can be the prohealing compound.

In another example, the prohealing compound can be a polysaccharide. In one aspect, the polysaccharide has at least one group, such as a carboxylic acid group or the salt or ester thereof that can react with a cycloaddition reactive moiety. In one example, the polysaccharide is a glycosaminoglycan (GAG). Any of the glycosaminoglycans described above can be used in this aspect. In another example, the prohealing compound is hyaluronan.

In some examples, the prohealing compound can be crosslinked with the polymeric composition. In one example, the prohealing compound and the polymeric composition each possess at least one cycloaddition reactive moiety, which then can react with a crosslinker having at least two cycloaddition reactive moieties. Any of the cycloaddition reactive moieties described herein can be used in this respect.

The anti-adhesion polymeric compositions can optionally contain a second prohealing compound. In one example, the second prohealing compound can be a growth factor. Any substance or metabolic precursor which is capable of promoting growth and survival of cells and tissues or augmenting the functioning of cells is useful as a growth factor. Examples of growth factors include, but are not limited to, a nerve growth promoting substance such as a ganglioside, a nerve growth factor, and the like; a hard or soft tissue growth promoting agent such as fibronectin (FN), human growth hormone (HGH), a colony stimulating factor, bone morphogenic protein, platelet-derived growth factor (PDGF), insulin-derived growth factor (IGF-I, IGF-II, transforming growth factor-alpha (TGF-alpha), transforming growth factor-beta (TGF-beta), epidermal growth factor (EGF), fibroblast growth factor (FGF), interleukin-1 (IL-1), vascular endothelial growth factor (VEGF) and keratinocyte growth factor (KGF), dried bone material, and the like; and antineoplastic agents such as methotrexate, 5-fluorouracil, adriamycin, vinblastine, cisplatin, tumor-specific antibodies conjugated to toxins, tumor necrosis factor, and the like. The amount of growth factor incorporated into the composite will vary depending upon the growth factor and prohealing compound selected as well as the intended end-use of the anti-adhesion polymeric composition.

Any of the growth factors disclosed in U.S. Pat. No. 6,534,591 B2, which is incorporated by reference in its entirety, can be used in this respect. In one example, the growth factor includes transforming growth factors (TGFs), fibroblast growth factors (FGFs), platelet derived growth factors (PDGFs), epidermal growth factors (EGFs), connective tissue activated peptides (CTAPs), osteogenic factors, and biologically active analogs, fragments, and derivatives of such growth factors. Members of the transforming growth factor (TGF) supergene family, which are multifunctional regulatory proteins. Members of the TGF supergene family include the beta transforming growth factors (for example, TGF-β1, TGF-β2, TGF-β3); bone morphogenetic proteins (for example, BMP-1, BMP-2, BMP-3, BMP-4, BMP-5, BMP-6, BMP-7, BMP-8, BMP-9); heparin-binding growth factors (for example, fibroblast growth factor (FGF), epidermal growth factor (EGF), platelet-derived growth factor (PDGF), insulin-like growth factor (IGF)); inhibins (for example, Inhibin A, Inhibin B); growth differentiating factors (for example, GDF-1); and Activins (for example, Activin A, Activin B, Activin AB).

Growth factors can be isolated from native or natural sources, such as from mammalian cells, or can be prepared synthetically, such as by recombinant DNA techniques or by various chemical processes. In addition, analogs, fragments, or derivatives of these factors can be used, provided that they exhibit at least some of the biological activity of the native molecule. For example, analogs can be prepared by expression of genes altered by site-specific mutagenesis or other genetic engineering techniques.

In another example, the addition of a crosslinker can be used to couple the polymeric composition with the prohealing compound. In one example, when the polymeric composition and the prohealing compound possess cycloaddition reactive moieties, a crosslinker having at least two cycloaddition reactive moieties can be used to couple the two compounds.

### Methods of Use

Any of the compounds, composites, compositions, and methods described herein can be used for a variety of uses. For example, the disclosed compositions can be used for drug delivery, small molecule delivery, wound healing, burn injury healing, and tissue regeneration. The disclosed compositions and methods are useful for situations which benefit from a hydrated, pericellular environment in which assembly of other matrix components, presentation of growth and differentiation factors, cell migration, or tissue regeneration are desirable.

The disclosed compositions and components can be placed directly in or on any biological system without purification. Examples of sites the disclosed compositions can be placed include, but are not limited to, soft tissue such as muscle or fat; hard tissue such as bone or cartilage; areas of tissue regeneration; a void space such as periodontal pocket; surgical incision or other formed pocket or cavity; a natural cavity such as the oral, vaginal, rectal or nasal cavities, the cul-de-sac of the eye, and the like; the peritoneal cavity and organs contained within, and other sites into or onto which the compounds can be placed including a skin surface defect such as a cut, scrape or burn area. Alternatively, the disclosed compositions can be used to extend the viability of damaged skin. The disclosed compositions can be biodegradable and naturally occurring enzymes can act to degrade them over time. The disclosed compositions can be “bioabsorbable” in that the disclosed compositions can be broken down and absorbed within the biological system, for example, by a cell, tissue and the like. Additionally, the disclosed compositions that have not been rehydrated can be applied to a biological system to absorb fluid from an area of interest.

The disclosed compositions can be used in a number of different surgical procedures. In one example, the disclosed compositions can be used in any of the surgical procedures disclosed in U.S. Pat. Nos. 6,534,591 B2 and 6,548,081 B2, which are incorporated by reference in their entireties. In one example, the disclosed compositions can be used in cardiosurgery and articular surgery; abdominal surgery where it is important to prevent adhesions of the intestine or the mesentery; operations performed in the urogenital regions where it is important to ward off adverse effects on the ureter and bladder, and on the functioning of the oviduct and uterus; and nerve surgery operations where it is important to minimize the development of granulation tissue. In surgery involving tendons, there is generally a tendency towards adhesion between the tendon and the surrounding sheath or other surrounding tissue during the immobilization period following the operation. In another example, the disclosed compositions can be used to prevent adhesions after laparascopic surgery, pelvic surgery, oncological surgery, sinus and craniofacial surgery, ENT surgery, or in procedures involving spinal dura repair.

In another example, the disclosed compositions can be used in ophthalmological surgery. In opthalmological surgery, a biodegradable implant could be applied in the angle of the anterior chamber of the eye for the purpose of preventing the development of synechiae between the cornea and the iris; this applies especially in cases of reconstructions after severe damaging events. Moreover, degradable or permanent implants are often desirable for preventing adhesion after glaucoma surgery and strabismus surgery.

In another example, the disclosed compositions can be used in the repair of tympanic membrane perforations (TMP). The tympanic membrane (TM) is a three-layer structure that separates the middle and inner ear from the external environment. These layers include an outer ectodermal portion composed of keratinizing squamous epithelium, an intermediate mesodermal fibrous component and an inner endodermal mucosal layer. This membrane is only 130 μm thick but provides important protection to the middle and inner ear structures and auditory amplification.

TMP is a common occurrence usually attributed to trauma, chronic otitis media or from PE tube insertion. Blunt trauma resulting in a longitudinal temporal bone fracture is classically associated with TMP. More common causes include a slap to the ear and the ill-advised attempt to clean an ear with a cotton swab (Q-Tip™) or sharp instrument.

Any of the disclosed compositions can be administered through the tympanic membrane without a general anesthetic and still provide enhanced wound healing properties. In one aspect, the disclosed compositions can be injected through the tympanic membrane using a cannula connected to syringe.

In another example, the disclosed compositions can be used as a postoperative wound barrier following endoscopic sinus surgery. Success in functional endoscopic sinus surgery (FESS) is frequently limited by scarring, which narrows or even closes the surgically widened openings. Spacers and tubular stents have been used to temporarily maintain the opening, but impaired wound healing leads to poor long-term outcomes. The use of any compounds, composites, and compositions described herein can significantly decrease scar contracture following maxillary sinus surgery.

In another example, the disclosed compositions can be used for the augmentation of soft or hard tissue. In another example, the disclosed compositions can be used to coat articles such as, for example, a surgical device, a prosthetic, or an implant (e.g., a stent). In another example, the disclosed compositions can be used to treat aneurisms.

The disclosed compositions can be used as a carrier and delivery device for a wide variety of releasable bioactive agents having curative or therapeutic value for human or non-human animals. Any of the bioactive agents described herein can be used in this respect. Many of these substances which can be carried by the disclosed compositions are discussed herein.

Included among bioactive agents that are suitable for incorporation into the disclosed compositions are therapeutic drugs, e.g., anti-inflammatory agents, anti-pyretic agents, steroidal and non-steroidal drugs for anti-inflammatory use, hormones, growth factors, contraceptive agents, antivirals, antibacterials, antifungals, analgesics, hypnotics, sedatives, tranquilizers, anti-convulsants, muscle relaxants, local anesthetics, antispasmodics, antiulcer drugs, peptidic agonists, sympathomimetic agents, cardiovascular agents, antitumor agents, oligonucleotides and their analogues and so forth. The bioactive agent is added in pharmaceutically active amounts.

The rate of drug delivery depends on the hydrophobicity of the molecule being released. For example, hydrophobic molecules, such as dexamethazone and prednisone are released slowly from the composition as it swells in an aqueous environment, while hydrophilic molecules, such as pilocarpine, hydrocortisone, prednisolone, cortisone, diclofenac sodium, indomethacin, 6∝-methyl-prednisolone and corticosterone, are released quickly. The ability of the compositions to maintain a slow, sustained release of steroidal anti-inflammatories makes the compounds described herein extremely useful for wound healing after trauma or surgical intervention.

In certain methods the delivery of molecules or reagents related to angiogenesis and vascularization are achieved. Disclosed are methods for delivering agents, such as VEGF, that stimulate microvascularization. Also disclosed are methods for the delivery of agents that can inhibit angiogenesis and vascularization, such as those compounds and reagents useful for this purpose disclosed in but not limited to U.S. Pat. Nos. 6,174,861 for “Methods of inhibiting angiogenesis via increasing in vivo concentrations of endostatin protein;” 6,086,865 for “Methods of treating angiogenesis-induced diseases and pharmaceutical compositions thereof;” 6,024,688 for “Angiostatin fragments and method of use;” 6,017,954 for “Method of treating tumors using O-substituted fumagillol derivatives;” 5,945,403 for “Angiostatin fragments and method of use;” 5,892,069 “Estrogenic compounds as anti-mitotic agents;” for 5,885,795 for “Methods of expressing angiostatic protein;” 5,861,372 for “Aggregate angiostatin and method of use;” 5,854,221 for “Endothelial cell proliferation inhibitor and method of use;” 5,854,205 for “Therapeutic antiangiogenic compositions and methods;” 5,837,682 for “Angiostatin fragments and method of use;” 5,792,845 for “Nucleotides encoding angiostatin protein and method of use;” 5,733,876 for “Method of inhibiting angiogenesis;” 5,698,586 for “Angiogenesis inhibitory agent;” 5,661,143 for “Estrogenic compounds as anti-mitotic agents;” 5,639,725 for “Angiostatin protein;” 5,504,074 for “Estrogenic compounds as anti-angiogenic agents;” 5,290,807 for “Method for regressing angiogenesis using o-substituted fumagillol derivatives;” and 5,135,919 for “Method and a pharmaceutical composition for the inhibition of angiogenesis” which are herein incorporated by reference for the material related to molecules for angiogenesis inhibition.

In one example, the bioactive agent is pilocarpine, hydrocortisone, prednisolone, cortisone, diclofenac sodium, indomethacin, 6∝-methyl-prednisolone, corticosterone, dexamethasone and prednisone. However, methods are also provided wherein delivery of a bioactive agent is for a medical purpose selected from the group of delivery of contraceptive agents, treating postsurgical adhesions, promoting skin growth, preventing scarring, dressing wounds, conducting viscosurgery, conducting viscosupplementation, engineering tissue.

In one example, the disclosed compositions can be used for the delivery of living cells to a subject. Any of the living cells described herein can be used in the respect. In one example, the living cells are part of a prohealing compound. In another example, the disclosed compositions can be used to support the growth of a variety of cells including, but not limited to, tumor cells, fibroblasts, chondrocytes, stem cells (e.g., embryonic, preadipocytes, mesenchymal, cord blood derived, bone marrow), epithelial cells (e.g., breast epithelial cells, intestinal epithelial cells), cells from neural lineages (e.g., neurons, astrocytes, oligodendrocytes, and glia), cells derived from the liver (e.g., hepatocytes), endothelial cells (e.g., vascular endothelial), cardiac cells (e.g., cardiac myocytes), muscle cells (e.g., skeletal or vascular smooth muscle cells), or osteoblasts. Alternatively, cells may be derived from cell lines or a primary source (e.g., human or animal), a biopsy sample, or a cadaver.

In one example, the disclosed compositions can be used for the delivery of growth factors and molecules related to growth factors. Any of the growth factors described herein are useful in this aspect. In one example, the growth factor is part of a prohealing compound.

In one example, described herein are methods for reducing or inhibiting adhesion of two tissues in a surgical wound in a subject by contacting the wound of the subject with any of the disclosed compositions. Not wishing to be bound by theory, it is believed that the disclosed compositions will prevent tissue adhesion between two different tissues (e.g., organ and skin tissue). It is desirable in certain post-surgical wounds to prevent the adhesion of tissues in order to avoid future complications.

The disclosed compositions provide numerous advantages. For example, the disclosed compositions can provide a post-operative adhesion barrier that is at least substantially resorbable and, therefore, does not have to be removed surgically at a later date. Another advantage is that the disclosed compositions are also relatively easy to use, can, in some instances, be sutured, and tend to stay in place after it is applied.

In another example, described herein are methods for improving wound healing in a subject in need of such improvement by contacting any of the disclosed compositions with a wound of a subject in need of wound healing improvement. Also provided are methods to deliver at least one bioactive agent to a subject in need of such delivery by contacting any of the disclosed compositions with at least one tissue capable of receiving said bioactive agent.

The disclosed compositions can be used for treating a wide variety of tissue defects in an animal, for example, a tissue with a void such as a periodontal pocket, a shallow or deep cutaneous wound, a surgical incision, a bone or cartilage defect, bone or cartilage repair, vocal fold repair, and the like. For example, the disclosed compositions can be in the form of a hydrogel film. The hydrogel film can be applied to a defect in bone tissue such as a fracture in an arm or leg bone, a defect in a tooth, a cartilage defect in the joint, ear, nose, or throat, and the like. The hydrogel film composed of the disclosed compositions can also function as a barrier system for guided tissue regeneration by providing a surface on or through which the cells can grow. To enhance regeneration of a hard tissue such as bone tissue, the hydrogel film can provide support for new cell growth that can replace the matrix as it becomes gradually absorbed or eroded by body fluids.

The disclosed compositions can be delivered onto cells, tissues, and/or organs, for example, by injection, spraying, squirting, brushing, painting, coating, and the like. Delivery can also be via a cannula, catheter, syringe with or without a needle, pressure applicator, pump, and the like. The disclosed compositions can be applied onto a tissue in the form of a film, for example, to provide a film dressing on the surface of the tissue, and/or to adhere to a tissue to another tissue or hydrogel film, among other applications.

In one example, the disclosed compositions can be administered via injection. For many clinical uses, when the disclosed compositions are in the form of a hydrogel film, injectable hydrogels can be used. An injectable hydrogel can be formed into any desired shape at the site of injury. Because the initial hydrogels can be sols or moldable putties, the systems can be positioned in complex shapes and then subsequently crosslinked to conform to the required dimensions. Also, the hydrogel would adhere to the tissue during gel formation, and the resulting mechanical interlocking arising from surface microroughness would strengthen the tissue-hydrogel interface. Further, introduction of an in situ-crosslinkable hydrogel could be accomplished using needle or by laparoscopic methods, thereby minimizing the invasiveness of the surgical technique.

The disclosed compositions can be used to treat periodontal disease, gingival tissue overlying the root of the tooth can be excised to form an envelope or pocket, and the composition delivered into the pocket and against the exposed root. The compounds, composites, and compositions can also be delivered to a tooth defect by making an incision through the gingival tissue to expose the root, and then applying the material through the incision onto the root surface by placing, brushing, squirting, or other means.

When used to treat a defect on skin or other tissue, the disclosed compositions can be in the form of a hydrogel film that can be placed on top of the desired area. In this aspect, the hydrogel film is malleable and can be manipulated to conform to the contours of the tissue defect.

The disclosed compositions can be applied to an implantable device such as a suture, claps, stents, prosthesis, catheter, metal screw, bone plate, pin, a bandage such as gauze, and the like, to enhance the compatibility and/or performance or function of an implantable device with a body tissue in an implant site. The disclosed compositions can be used to coat the implantable device. For example, the disclosed compositions could be used to coat the rough surface of an implantable device to enhance the compatibility of the device by providing a biocompatible smooth surface which reduces the occurrence of abrasions from the contact of rough edges with the adjacent tissue. The disclosed compositions can also be used to enhance the performance or function of an implantable device. For example, when the disclosed compositions are a hydrogel film, the hydrogel film can be applied to a gauze bandage to enhance its compatibility or adhesion with the tissue to which it is applied. The hydrogel film can also be applied around a device such as a catheter or colostomy that is inserted through an incision into the body to help secure the catheter/colostomy in place and/or to fill the void between the device and tissue and form a tight seal to reduce bacterial infection and loss of body fluid.

In one example, the disclosed compositions that comprise, for example, PLUORONICS™, can couple to GAGs such as, for example, hyaluronan or heparin, and self-assemble into hydrogels. Alternatively, solutions of the disclosed compositions and GAGs can be coated on a hydrophobic surface such as, for example, a medical device. For example, heparin can be coupled with an hydrophilic polymer comprising a PLUORONIC™, wherein the resultant gel possesses desirable growth-binding factor capabilities but does not possess anti-coagulant properties associated with heparin. Not wishing to be bound by theory, the PLUORONIC™ portion of the hydrogel can prevent coagulation, which is undesirable side-effect of heparin.

It is understood that the disclosed compositions can be applied to a subject in need of tissue regeneration. For example, cells can be incorporated into the disclosed compositions herein for implantation. Examples of subjects that can be treated with the disclosed compositions include mammals such as mice, rats, cows or cattle, horses, sheep, goats, cats, dogs, and primates, including apes, chimpanzees, orangutans, and humans. In another aspect, the disclosed compositions can be applied to birds.

When being used in areas related to tissue regeneration such as wound or burn healing, it is not necessary that the disclosed compositions and methods eliminate the need for one or more related accepted therapies. It is understood that any decrease in the length of time for recovery or increase in the quality of the recovery obtained by the recipient of the disclosed compositions and methods has obtained some benefit. It is also understood that some of the disclosed compositions and methods can be used to prevent or reduce fibrotic adhesions occurring as a result of wound closure as a result of trauma, such surgery. It is also understood that collateral affects provided by the disclosed compositions and methods are desirable but not required, such as improved bacterial resistance or reduced pain etc.

In one example, the disclosed compositions can be used to prevent airway stenosis. Subglottic stenosis (SGS) is a condition affecting millions of adults and children world-wide. Causes of acquired SGS range from mucosal injury of respiratory epithelia to prolonged intubation. Known risk factors of SGS in intubated patients include prolonged intubation, high-pressure balloon cuff, oversized endotracheal (ET) tube, multiple extubations or re-intubations, and gastro-esophageal reflux. There are also individuals in whom stenosis develops as a result of surgery, radiation, autoimmune disease, tumors, or other unexplained reasons.

While very diverse, the etiologies of SGS all have one aspect in common, narrowing of the airway resulting in obstruction. This narrowing most commonly occurs at the level of the cricoid cartilage due to its circumferential nature and rigidity. Such etiologies have been found in various SGS models: activation of chondrocytes and formation of fibrous scar, infiltration of polymorphonuclear leukocytes and chronic inflammatory cells with squamous metaplasia, and morphometric changes in airway lumen. Each presents a problem requiring immediate attention.

In another example, any of the disclosed compositions can be used as a 3-D cell culture. In one example, the hydrogel can be lyophilized to create a porous sponge onto which cells may be seeded for attachment, proliferation, and growth. It is contemplated that miniarrays and microarrays of 3-D hydrogels or sponges can be created on surfaces such as, for example, glass, and the resulting gel or sponge can be derived from any of the compounds or compositions described herein. The culture can be used in numerous embodiments including, but not limited to, determining the efficacy or toxicity of experimental therapeutics.

### Kits

In a further aspect, disclosed herein is a kit including (1) a hydrophilic polymer comprising at least one cycloaddition reactive moiety and (2) a crosslinker comprising at least two cycloaddition reactive moieties. The kit can also comprise a catalyst. In some examples, the hydrophilic polymer can be any hydrophilic polymer disclosed herein. The cycloaddition reactive moiety on the hydrophilic polymer can also be any such moiety disclosed herein. Further, the crosslinker and its cycloaddition reactive moieties can be any of those disclosed herein. Use of the kit generally involves admixing components (1) and (2) together under cycloaddition conditions. Components (1) and (2) can be added in any order. For example, the hydrophilic polymer and crosslinker can be in separate containers (e.g., syringes or spray cans), with the contents being mixed using when they are expelled together (e.g., by syringe-to-syringe techniques or spraying through the nozzle of a spray can) just prior to delivery to the subject.

In another example, the polymeric composition and anti-adhesion and/or prohealing compounds can be used as a kit. For example, the polymeric composition and anti-adhesion and/or prohealing compounds are in separate syringes, with the contents being mixed using syringe-to-syringe techniques just prior to delivery to the subject. In this example, the polymeric composition and anti-adhesion and/or prohealing compounds can be extruded from the opening of the syringe by an extrusion device followed by spreading the mixture via spatula.

In another example, the polymeric composition and the anti-adhesion and/or prohealing compounds are in separate chambers of a spray can or bottle with a nozzle or other spraying device. In this example, the first compound and anti-adhesion and/or prohealing compounds do not actually mix until they are expelled together from the nozzle of the spraying device.

### EXAMPLES

The following examples are put forth so as to provide those of ordinary skill in the art with a complete disclosure and description of how the compounds, compositions, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

### Example 1

### Synthesis of Azide-Functionalized Polymer

First, azidotoluic acid was synthesized following the methods of Zhou and Fahrni (J. Am. Chem. Soc. 2004, 126, 8862-3). Bromotoluic acid was reacted with excess sodium azide in absolute ethanol at reflux for 24 hours. Once cooled, an equal volume of water was added to the reaction, and then concentrated HCl was added to precipitate out the product. Precipitation was brought to completion by chilling overnight at 4° C. The product was then filtered off, washed with water, and dried overnight in vacuo. Purified product was confirmed by ¹H NMR and ¹³C NMR. Yields commonly ranged from 60 to 80%.

Next, purified azidotoluic acid was used to functionalize 4-arm poly(ethylene glycol) following esterification methods similar to Blankemeyer-Menge et al. (Tetrahedron 1990, 31, 1701-4). Briefly, a mixture of 10 equivalents (eq.) azidotoluic acid and 10 eq. methylimidazole (MeIm) in dry dichloromethane was added to 10 eq. MSNT by syringe. This mixture was then added to 1 eq. 4-arm PEG (MW ˜10,000 Da) dissolved in dry dichloromethane and allowed to stir at room temperature for 48 hours under N₂ (gas). Following 48 hours, the reaction was thrice washed with an aqueous solution of 100 mM Na₂PO₄ and 1 M Na₂SO₄ (pH 7). The organic layer was then dried over Na₂SO₄, precipitated in hexane, concentrated by rotary evaporation, and dried overnight in vacuo. Purified product was confirmed by ¹H NMR and MALDI mass spectrometry. Yields commonly ranged from 66 to 76%.

### Example 2

### Synthesis of Dialkyne/Dialkene Crosslinkers

Dipentynoic ester PEG was synthesized using an esterification method similar to Hassner and Alexanian (Tetrahedron Lett. 1978, 4475-8). 2.2 eq. of pentynoic acid was dissolved in dry dichloromethane. To this solution, 2.2 eq. diisopropylcarbodiimide (DIC) and 0.2 eq. pyrrolidinopyridine (PP) was added, followed by 1 eq. PEG (W 400 Da). The reaction was run 24 hours at room temperature. Following 24 hours, the reaction was thrice washed with an aqueous solution of 100 mM Na₂PO₄ and 1 M Na₂SO₄ (pH 7). The organic layer was then dried over Na₂SO₄, concentrated by rotary evaporation, and dried overnight in vacuo. Purified product was confirmed by ¹H NMR. Calculated yields were commonly about 76%.

Dipropiolic amide PEG was synthesized using a symmetric anhydride method. 2 eq. propiolic acid was added dropwise to 2.4 eq. DIC dissolved in dry DCM while under N₂ (gas) and chilled in a water-ice bath. Next, 1 eq. ethylene dioxy bisethyl amine dissolved in dry DCM was added to the reaction 10 minutes later, still under N₂ (gas) and chilled in a water-ice bath. Following stirring at 0° C. for 1 hour, the reaction was continued at room temperature overnight. The product was purified by liquid chromatography using 100% chloroform, giving a yield of 80%, which was confirmed by ¹H NMR and ESI(+) mass spectrometry.

Dinorbornene ester PEG was synthesized using a HOBT-ester method. 3 eq. norbornene carboxylic acid and 3 eq. HOBT were dissolved in dry DCM, and chilled in a chloroform-liquid nitrogen bath. 3 eq. DIC were then added dropwise to the chilled solution, and then allowed to run overnight at room temperature. Following 24 hours, the reaction was again chilled to −60° C., and a mixture of 1 eq. tetraethylene glycol and 2 eq. triethylamine in dry DCM was added dropwise. The reaction was allowed to warm to room temperature and then stirred overnight. The product, which was confirmed by ¹H NMR and ESI(+) mass spectrometry, was purified by filtering off any precipitate, running the solution through a disk of silica, concentrating by rotary evaporation, and drying in vacuo.

### Example 3

### Copper-Catalyzed Hydrogel Formation

1 eq. azide-functionalized 4-arm PEG polymer and 2 eq. dipentynoic ester PEG crosslinker were dissolved in water separately using molar concentrations of 0.0169 M and 0.0338 M, respectively. Copper(I) catalyst, in either the form 0.1 eq. copper(II) sulfate plus 1 eq. sodium ascorbate or 0.1 eq. copper(II) sulfate plus 1 eq. sodium ascorbate and 0.1 eq. triazole ligand (such as tris(ethylacetatatriazole) amine) (Zhou and Fahrni, J. Am. Chem. Soc. 2004, 126, 8862-3; Chan et al., Org. Lett. 2004, 6, 2853-5) was then added to either polymer before mixing. Immediately upon catalyst addition, the two liquid components were mixed and stored at 37° C. Hydrogels formed under all conditions described with the fastest gelation time (less than 15 minutes) occurring when the catalyst was added to the dialkyne crosslinker first. This result is supported by the previously-suggested mechanism for copper catalyzed click chemistry, in which Cu(I) binds to the terminal alkyne, then allowing the azide to attack (Rostovtsev et al., Angew. Chem. Int. Ed. 2002, 41, 2596-9).

### Example 4

### Catalyst-Free Hydrogel Formation

1 eq. azide-functionalized 4-arm PEG polymer and 2 eq. dipropiolic amide PEG crosslinker were dissolved in water using molar concentrations of 0.169 M and 0.338 M, respectively. The reactions were vortexed for 30-60 seconds, or until fully dissolved, and then stored at 37° C. Hydrogels formed within 48 hours of mixing.

### Prophetic Example 5

### Synthesis of Strain-Promoted Alkyne Crosslinkers

A strain-promoted alkyne crosslinker, such as dicyclooctyne ester PEG, can also be used (see FIG. 4). A cyclooctyne-functionalized carboxylic acid can be synthesized based on the synthetic scheme of Agard et al. (Agard et al., J. Am. Chem. Soc. 2004, 126, 15046-7). This cycloaddition reactive moiety can be coupled to a small MW PEG via esterification in a manner similar to that used in Example 1 for dipropiolic amide PEG and dinorbornene PEG.

### Prophetic Example 6

### Biocompatibility of Click-Based Gelation in the Presence of Cells

The cytotoxicity of click-based hydrogels formed in the presence of cells can be evaluated. Experiments can be performed by 1) mixing the two-part polymer systems (with and without catalyst) and immediately (prior to gelation) applying the mixture to the surface of cell monolayers, and 2) suspending cells in one of the two polymer parts prior to mixing and gelation. These studies can be performed using live/dead cytotoxicity assays on L929 mouse fibroblasts. Cell culture media can replace water as the gelation solvent.

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

1. A polymeric composition, comprising: a hydrophilic polymer residue and a crosslinker residue, wherein the hydrophilic polymer residue is bonded to the crosslinker residue with a moiety formed from a cycloaddition reaction, and wherein the polymeric composition is not a polyacrylamide crosslinked with a photo activated 2+2 cycloaddition reaction.

2. The polymeric composition of claim 1, wherein the polymeric composition comprises one or more moieties having Formula I:
L-(Z-R)ₙ (I)
where L is the crosslinker residue, R is the hydrophilic polymer residue, Z is the moiety formed from the cycloaddition reaction, and n is at least 2.

3. The polymeric composition of claim 1, wherein the hydrophilic polymer residue is bonded to the crosslinker residue with a moiety formed from a 3+2 cycloaddition reaction.

4. The polymeric composition of claim 1, wherein the hydrophilic polymer residue is bonded to the crosslinker residue with a moiety formed from a 2+2 cycloaddition reaction.

5. The polymeric composition of claim 1, wherein the moiety formed from a cycloaddition reaction is a triazole moiety or a triazoline moiety.

6. (canceled)

7. The polymeric composition of claim 1, wherein the hydrophilic polymer residue comprises a homopolymer.

8. The polymeric composition of claim 1, wherein the hydrophilic polymer residue comprises a block, graft, or graft comb copolymer.

9. (canceled)

10. The polymeric composition of claim 1, wherein the hydrophilic polymer residue comprises polyethylene oxide or polypropylene oxide.

11. (canceled)

12. The polymeric composition of claim 1, wherein the hydrophilic polymer residue comprises a multi-armed polymer.

13. (canceled)

14. The polymeric composition of claim 1, wherein the hydrophilic polymer residue comprises a dendrimer.

15. (canceled)

16. (canceled)

17. The polymeric composition of claim 1, wherein the hydrophilic polymer residue comprises a triblock polymer of poly(ethylene oxide)-poly(propylene oxide)-poly(ethylene oxide).

18-25. (canceled)

26. The polymeric composition of claim 1, wherein the crosslinker residue is a residue of a di-, tri-, tetra-, penta-, hexa-, hepta-, octa-, nona-, or deca-valent crosslinker.

27. (canceled)

28. The polymeric composition of claim 1, wherein the crosslinker residue comprises a C₁-C₆ branched or straight-chain alkyl.

29. The polymeric composition of claim 1, wherein the crosslinker residue comprises a C₁-C₆ branched or straight-chain alkoxy.

30. The polymeric composition of claim 1, wherein the crosslinker residue comprises a methoxymethyl, methoxyethyl, methoxypropyl, methoxybutyl, ethoxymethyl, ethoxyethyl, ethoxypropyl, propoxymethyl, propoxyethyl, methylaminomethyl, methylaminoethyl, methylaminopropyl, methylaminobutyl, ethylaminomethyl, ethylaminoethyl, ethylaminopropyl, propylaminomethyl, propylaminoethyl, methoxymethoxymethyl, ethoxymethoxymethyl, methoxyethoxymethyl, or methoxymethoxyethyl.

31. The polymeric composition of claim 1, wherein the crosslinker residue comprises the formula —(OCH₂CH₂)ₘ—, wherein m is from 2 to 10.

32. (canceled)

33. The polymeric composition of claim 1, wherein the polymeric composition comprises a hydrogel.

34. The polymeric composition of claim 1, wherein the polymeric composition further comprises one or more bioactive agents.

35. The polymeric composition of claim 34, wherein the bioactive agent comprises a growth factor, an anti-inflammatory agent, an anti-cancer agent, an analgesic, an anti-infection agent, an anti-viral agent, a hormone, an antibody, or a therapeutic protein.

36-40. (canceled)

41. The polymeric composition of claim 1, wherein the polymeric composition is biodegradable.

42. (canceled)

43. A method of making a polymeric composition, comprising: contacting a hydrophilic polymer comprising one or more cycloaddition reactive moieties with a crosslinker comprising two or more cycloaddition reactive moieties, wherein the cycloaddition reactive moieties undergo a cycloaddition reaction to provide the polymeric composition, and wherein the polymeric composition is not a polyacrylamide crosslinked with a photoactive 2+2 cycloaddition reaction.

44. The method of claim 43, wherein the cycloaddition reactive moieties under a 3+2 cycloaddition reaction.

45. The method of claim 43, wherein the cycloaddition reactive moieties under a 2+2 cycloaddition reaction.

46. (canceled)

47. The method of claim 43, wherein the hydrophilic polymer and crosslinker are contacted at a pH of from about 4 to about 8.

48. The method of claim 43, wherein the hydrophilic polymer and crosslinker are contacted in aqueous media or in biological fluids.

49. (canceled)

50. The method of claim 43, wherein the hydrophilic polymer and crosslinker are contacted at from about 25° C. to about 37° C.

51. The method of claim 43, wherein the hydrophilic polymer and crosslinker are contacted in the presence of cells, biomolecules, tissues, or salts.

52. The method of claim 43, wherein the hydrophilic polymer and crosslinker are contacted in the presence of a bioactive agent, an anti-adhesion compound, or a prohealing compound.

53. (canceled)

54. The method of claim 43, wherein the hydrophilic polymer and crosslinker are contacted in the presence of a copper catalyst.

55. (canceled)

56. The method of claim 54, wherein the catalyst comprises copper sulfate, copper bromide, or copper iodide.

57. The method of claim 54, wherein the catalyst is further combined with a reducing agent.

58. (canceled)

59. The method of claim 54, wherein the catalyst is further combined with a stabilizing ligand.

60. The method of claim 59, wherein the stabilizing ligand is a tris-triazolyl compound.

61. The method of claim 43, wherein the hydrophilic polymer comprises 1, 2, 3, 4, 5, 6, 7, 8, 9, or 10 cycloaddition reactive moieties.

62. The method of claim 61, wherein the cycloaddition reactive moiety comprises a dipolarophile.

63. (canceled)

64. The method of claim 62, wherein the dipolarophile comprises an alkene or an alkyne.

65. The method of claim 61, wherein the cycloaddition reactive moiety comprises a 1,3-dipolar group.

66. The method of claim 65, wherein the 1,3-dipolar group comprises an azide.

67. The method of claim 65, wherein the 1,3-dipolar group comprises a diazoalkane, nitrous oxide, nitrile ylide, nitrile imine, nitrile oxide, azomethine ylide, azomethine imine, nitrone, azimine, azoxy group, nitro group, carbonyl ylide, carbonyl imine, carbonyl oxide, nitrosimine, nitrosoxide, or ozone.

68. The method of claim 61, wherein the hydrophilic polymer comprises a 1,3-dipolar group and a dipolarophile.

69. The method of claim 43, wherein the crosslinker comprises 1, 2, 3, 4, 5, 6, 7, 8, 9, or 10 cycloaddition reactive moieties.

70. The method of claim 69, wherein the cycloaddition reactive moiety comprises a dipolarophile.

71. (canceled)

72. The method of claim 70, wherein the dipolarophile comprises an alkene or an alkyne.

73. The method of claim 69, wherein the cycloaddition reactive moiety comprises a 1,3-dipolar group.

74. The method of claim 73, wherein the 1,3-dipolar group comprises an azide.

75. The method of claim 73, wherein the 1,3-dipolar group comprises a diazoalkane, nitrous oxide, nitrile ylide, nitrile imine, nitrile oxide, azomethine ylide, azomethine imine, nitrone, azimine, azoxy group, nitro group, carbonyl ylide, carbonyl imine, carbonyl oxide, nitrosimine, nitrosoxide, or ozone.

76. The method of claim 69, wherein the crosslinker comprises a 1,3-dipolar group and a dipolarophile.

77. The method of claim 43, wherein the cycloaddition reactive moiety on the hydrophilic polymer comprises a 1,3-dipolar group and the cycloaddition reactive moiety on the crosslinker comprises a dipolarophile.

78. The method of claim 77, wherein the cycloaddition reactive moiety on the hydrophilic polymer comprises an azide and the cycloaddition reactive moiety on the crosslinker comprises an alkyne.

79. The method of claim 43, wherein the cycloaddition reactive moiety on the hydrophilic polymer comprises a dipolarophile and the cycloaddition reactive moiety on the crosslinker comprises a 1,3-dipolar group.

80. The method of claim 79, wherein the cycloaddition reactive moiety on the hydrophilic polymer comprises an alkyne and the cycloaddition reactive moiety on the crosslinker comprises an azide.

81-84. (canceled)

85. A pharmaceutical composition comprising a bioactive agent and the polymeric composition of claim 1.

86. A method for improving wound healing in a subject in need of such improvement, comprising contacting the wound of the subject with the polymeric composition of claim 1.

87-101. (canceled)

102. An article coated with the polymeric composition of claim 1.

103. The article of claim 102, wherein the article is a suture, a clap, stent, a prosthesis, a catheter, a metal screw, a bone plate, a pin, or a bandage.

104. (canceled)
